(19) United States
(12) Patent Application Publication
(10) Pub. No.: US 2025/0304713 A1
(43) Pub. Date: Oct. 2, 2025

(54) COMBINATION IMMUNOTHERAPY COMPOSITIONS AND METHODS OF USE THEREOF

(71) Applicant: THE PENN STATE RESEARCH FOUNDATION, University Park, PA (US)

(72) Inventors: Hong-Gang WANG, University Park, PA (US); Xiaoming LIU, University Park, PA (US); Carson A. WILLS, University Park, PA (US)

(21) Appl. No.: 18/842,253

(22) PCT Filed: Mar. 1, 2023
(86) PCT No.: PCT/US2023/063444

§ 371 Date: Aug. 28, 2024

Related U.S. Application Data
(60) Provisional application No. 63/315,257, filed on Mar. 1, 2022.

(51) Int. Cl. C07K 16/30; A61K 31/4709; A61K 31/56; A61K 38/18; A61K 38/19; A61K 45/06; A61P 35/04

(52) U.S. Cl. CPC C07K 16/30; A61K 31/4709; A61K 31/56; A61K 38/18; A61K 38/19; A61K 45/06; A61P 35/04

(57) ABSTRACT

The present disclosure relates to anti-GD2 and/or famesyltransferase inhibitor compositions for use in methods for treating a cancer, for example, neuroblastoma. Also provided is a method of preventing neuroblastoma metastasis, comprising administering to a subject a therapeutically effective amount of an anti-GD2 immunotherapy and a farnesyltransferase inhibitor. Further provided are compositions comprising tipifarnib and/or dinutuximab and methods of use thereof.

### CROSS-REFERENCE TO RELATED APPLICATIONS

This application claims the benefit of U.S. Provisional Application No. 63/315,257, filed Mar. 1, 2022, which is expressly incorporated herein by reference in its entirety.

### REFERENCE TO A SEQUENCE LISTING

The Sequence Listing submitted Mar. 1, 2023 as an XML file named “11196-075W01_Sequence_Listing.xml,” created on Feb. 28, 2023, and having a size of 5466 bytes is hereby incorporated by reference pursuant to 37 C.F.R. § 1.834.

### BACKGROUND OF THE INVENTION

Neuroblastoma is the most common extracranial solid tumor in children, accounting for approximately 6% of all pediatric malignancies but more than 10% of all childhood cancer-related deaths. The standard treatment regimen for patients with high-risk neuroblastoma includes multi-agent chemotherapy, surgery, autologous stem cell transplantation, radiotherapy, and maintenance therapy. Despite multimodal treatment, the five-year overall survival rate for patients with high-risk disease is only around 50%.

The recent incorporation of dinutuximab and immunostimulatory agents [granulocyte-macrophage colony-stimulating factor (GM-CSF) and interleukin-2 (IL2)] to the maintenance therapy for patients with high-risk neuroblastoma has substantially improved patient outcomes. Dinutuximab is a chimeric monoclonal antibody against the disialoganglioside GD2, which is expressed on the outer leaflet of the plasma membrane of peripheral neurons, skin melanocytes and the central nervous system and is ubiquitously present on tumors of neuroectodermal origin including most neuroblastomas. Tumor-bound anti-GD2 antibodies recruit immune effector cells to trigger Fc-receptor-mediated killing by both complement-mediated cytotoxicity and antibody-dependent cell-mediated cytotoxicity (ADCC).

Despite its relative success, more than 40% of neuroblastoma patients fail to respond or develop resistance to anti-GD2 therapy. Moreover, although anti-GD2 immunotherapy is highly effective against minimal residue disease in bone marrow (BM), it is much less efficient for targeting solid tumors. However, the factors underlying therapeutic failure and resistance to anti-GD2 immunotherapy remain unknown.

Small extracellular vesicles (sEVs) have recently emerged as critical regulators of tumor growth, metastasis and cancer progression. The 30-150 nm vesicles are secreted by almost all cell types through outward budding of the plasma membrane or direct fusion of multivesicular bodies with the plasma membrane. Notably, sEVs contain biologically active molecules capable of modulating the extracellular environment and immune system. Recent studies have found that tumor-derived sEVs play an important role in promoting resistance to immunotherapy by interacting with immune effector cells and suppressing the host immune system. NK cells, which express the receptor FcgRIIIa (CD16), are the major effector cells for anti-GD2 immunotherapy and utilize ADCC to target neuroblastoma cells. Tumor-derived sEVs have been shown to attenuate ADCC in vitro by inhibiting the binding of antibodies to tumor cells (15). Moreover, tumor-derived sEVs have been shown to dysregulate NK cell function and induce NK cell exhaustion. However, whether tumor-derived sEVs regulate resistance to anti-GD2 monoclonal antibody immunotherapy in vivo remains unclear.

### SUMMARY OF THE INVENTION

The present disclosure relates to synergistically-effective anti-GD2/farnesyltransferase inhibitor compositions for neuroblastoma, including high-risk neuroblastoma.

Disclosed herein are compositions comprising an anti-GD2 immunotherapy and a farnesyltransferase inhibitor, including those wherein the farnesyltransferase inhibitor comprises tipifarnib and/or the anti-GD2 immunotherapy comprises dinutuximab.

Also disclosed are methods of treating a cancer comprising administering to a subject in need thereof an anti-GD2 immunotherapy and a farnesyltransferase inhibitor, including those wherein the cancer is neuroblastoma or high-risk neuroblastoma.

In some embodiments, the anti-GD2 immunotherapy is dinutuximab and/or the farnesyltransferase inhibitor is tipifarnib.

In other embodiments, the methods of treating a cancer further comprise administering a therapeutically effective amount of at least one or more active agents or a support care therapy selected from the group comprising: a DNA-hypomethylating agent, therapeutic antibody that specifically binds to a cancer antigen, a hematopoietic growth factor, a cytokine, an antibiotic, a cox-2 inhibitor, an immunomodulatory agent, an anti-thymocyte globulin, an immunosuppressive agent, and a corticosteroid.

In some embodiments, the administration of the therapeutically effective amount of the anti-GD2 immunotherapy and the farnesyltransferase inhibitor provides an increase in immune cell tumor infiltration, a decrease in tumor-associate macrophages, an increase in tumor-infiltrating natural killer (NK) cells, an increase in splenic NK cell maturation, an increase in NK cell-mediated antibody-dependent cellular cytotoxicity, a decrease of immunosuppressive effects of neuroblastoma-derived sEVs, or a decrease of formation of CD11b+Ly6C(high)Ly6G(low) cells.

In some embodiments, the subject comprises one or more cell markers corresponding to low expression of NKG2D and/or RAS/MAPK signal pathway deficiencies.

In some embodiments, the neuroblastoma is a GD2 inhibitor-refractory neuroblastoma (GIRN).

Also disclosed herein are methods of preventing neuroblastoma metastasis, comprising administering to a subject a therapeutically effective amount of an anti-GD2 immunotherapy and a farnesyltransferase inhibitor, including those wherein the anti-GD2 immunotherapy is dinutuximab and/or wherein the farnesyltransferase inhibitor is tipifarnib.

In some embodiments, the method of preventing neuroblastoma metastasis further comprises administering a therapeutically effective amount of at least one or more active agents or a support care therapy selected from the group consisting of a DNA-hypomethylating agent, therapeutic antibody that specifically binds to a cancer antigen, a hematopoietic growth factor, a cytokine, an antibiotic, a cox-2 inhibitor, an immunomodulatory agent, an anti-thymocyte globulin, an immunosuppressive agent, and a corticosteroid.

In other embodiments, the method of preventing neuroblastoma metastasis further comprises administration of the therapeutically effective amount of the anti-GD2 immunotherapy and the farnesyltransferase inhibitor provides an increase in immune cell tumor infiltration, a decrease in tumor-associate macrophages, an increase in tumor-infiltrating natural killer (NK) cells, an increase in splenic NK cell maturation, an increase in NK cell-mediated antibody-dependent cellular cytotoxicity, a decrease of immunosuppressive effects of neuroblastoma-derived sEVs, or a decrease of formation of CD11b+Ly6C(high)Ly6G(low) cells. The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects described below.

FIGS. 1A-1G. Neuroblastoma-derived sEVs induce resistance to dinutuximab in vivo. FIG. 1A, Representative electron microscopy images of sEVs isolated from 9464D-GD2 cells. Scale bar, 200 nm. FIG. 1B, Immunoblot of positive and negative sEV markers in whole cell lysate (WCL) and purified sEVs isolated from 9464D-GD2 cells. All lanes loaded with equal amount of protein. FIG. 1C, Representative size distribution and concentration of sEVs isolated from 9464D-GD2 cells quantified by NTA. FIG. 1D, Experimental design. C57BL/6 mice were subcutaneously inoculated with 1×10⁶ 9464D-GD2 cells. One-week post-inoculation, tumor-bearing mice began receiving tail-vein injections of PBS, dinutuximab (1.25 mg/kg) and/or 9464D-GD2-derived sEVs (20 μg) twice per week. On day 30, tumors were harvested for analysis. FIG. 1E, Quantification of tumor volume at the indicated time points. Mean±SEM. PBS, n=11; sEVs, n=10; dinutuximab, n=12; dinutuximab+sEVs, n=11. FIG. 1F, Quantification of tumor volume and weight from indicated treatment groups on day 30. Mean±SEM, n as in (E). Two-tailed unpaired t-test. *, p<0.05; **, p<0.01. G, Representative images of tumors from indicated treatment groups on day 30. Scale bar, 1 cm.

FIGS. 2A-2F. Neuroblastoma-derived sEVs inhibit dinutuximab-induced NK cell tumor infiltration and recruit tumor-associated macrophages. FIG. 2A, Top 20 biological processes identified by GO enrichment analysis of 9464D-GD2 tumors treated with dinutuximab plus sEVs versus dinutuximab alone as described in FIG. 2A. FIG. 2B-FIG. 2C, GSEA enrichment plots of two clusters enriched in dinutuximab plus sEVs tumors were: FIG. 2B negative regulation of lymphocyte activation and FIG. 2C myeloid leukocyte mediated immunity. FIG. 2D, GSEA enrichment plot for the extrinsic apoptotic signaling pathway. FIG. 2E-FIG. 2F, C57BL/6 mice were inoculated with 9464D-GD2 cells and treated as described in FIG. 1D. FIG. 2E, Quantification and representative flow cytometry plots of tumor-infiltrating NK cells (NK1.1+CD3−; top panel) and tumor-associated macrophages (TAMs) (CD11b+F4/80+; bottom panel) in 9464D-GD2 tumors isolated from mice on day 30 in the indicated treatment groups. Mean±SEM. n=7 per group. Student's t-test. *, p<0.05; **, p<0.01; * * *, p<0.001. FIG. 2F, Quantification and representative flow cytometry plots of NK cells (NK1.1+CD3−) isolated from the blood of tumor-bearing mice on day 30 in the indicated treatment groups. Mean SEM. n=7 per group. Student's t-test. *, p<0.05; **, p<0.01; ***, p<0.001.

FIGS. 3A-3F. Neuroblastoma-derived sEVs modulate NK cell subpopulations in vivo and NK cell-mediated ADCC in vitro. FIG. 3A, Schematic of NK cell subpopulations. DN, double negative. DP, double positive. FIG. 3B, Representative flow cytometry plots of splenic NK cell subpopulations isolated from 9464D-GD2 tumor-bearing mice receiving the indicated treatments as described in FIG. 1D. FIG. 3C, Quantification of the percentage of immature NK cells (CD27+CD11b−; left panel) and mature NK cells (CD27−CD11b+; right panel) subpopulations in the spleen of 9464D-GD2 tumor-bearing mice receiving the indicated treatments as described in FIG. 1D. Mean±SEM, n=7 per group. Student's t-test. *, p<0.05; **, p<0.01; ***, p<0.001. FIG. 3D-FIG. 3G, Human (IMR32) or murine (9464D-GD2) neuroblastoma cells were treated as indicated in the presence or absence of NK92-CD16-EGFP cells (NK) and monitored for viability in the presence of the cell impermeant nucleic acid stain YOYO-3 using the IncuCyte S3 Live-Cell Analysis System. Viable tumor cells (YOYO3−EGFP−) were quantified using the Cell-by-Cell Analysis module. FIG. 3D, Kinetic analysis for IMR32 cells. Mean±SEM, n=6. Quantification of the percentage of viable tumor cells normalized to no treatment control. FIG. E IMR32 at 24 h, n=6. FIG. F 9464D-GD2 at 24 h, n=4. sEVs were derived from the respective neuroblastoma cell lines. Mean±SD. One-way ANOVA with Tukey's/Sidak's post hoc tests. *, p<0.05; **, p<0.01; ***, p<0.001.

FIGS. 4A-4H. Inhibition of sEV secretion by tipifarnib sensitizes 9464D-GD2 tumors to dinutuximab. FIG. 4A, Representative electron microscopy images of sEVs isolated from 9464D-GD2 cells treated with DMSO or 0.1 μM tipifarnib for 48 h. Scale bar, 200 nm. FIG. 4B-FIG. 4C, NTA analysis of sEVs isolated from 9464D-GD2 cells treated with DMSO or 0.1 μM tipifarnib for 48 h. FIG. 4B Size distribution. FIG. 4C Particle concentration. Mean±SD, n=4. Student's t-test. * * *, p<0.001. FIG. 4D, Experimental design for E-H. C57BL/6 mice were subcutaneously inoculated with 1×10⁶ 9464D-GD2 cells. One-week post-inoculation, tumor-bearing mice began receiving tail-vein injections of PBS or dinutuximab (1.25 mg/kg) twice per week combined with oral administration of vehicle or tipifarnib (25 mg/kg/half day) twice per day. On day 24, tumors were harvested for analysis. FIG. 4E, Quantification of sEV protein content isolated from serum of mice receiving the indicated treatments. Mean±SEM, n=4 mice per group. Student's t-test. *, p<0.05. FIG. 4F, Quantification of tumor volume at the indicated time points. Mean±SEM, n=17 per treatment group. Data represent two independent experiments. FIG. 4G, Representative images of tumors from indicated treatment groups on day 24. Scale bar, 1 cm. FIG. 4H, Quantification of tumor volume and weight from indicated treatment groups on day 24. Mean±SEM, n=17 per treatment group. Student's t-test. *, p<0.05; * *, p<0.01; * * *, p<0.001. Data represent two independent experiments.

FIGS. 5A-5F. Tipifarnib remodels the tumor microenvironment and reverses the systemic immune suppression induced by neuroblastoma-derived sEVs to enhance the efficacy of dinutuximab. FIG. 5A, Quantification of tumor-infiltrating NK cells (NK1.1+CD3−; n=15; top panel), tumor-associated T-cells (CD3+NK1.1−; n=15; middle panel) and tumor-associated macrophages (TAM) (CD11b+F4/80+; n=17; bottom panel) in 9464D-GD2 tumors isolated from mice in the indicated treatment groups on day 24 as described in FIG. 4D. Mean±SEM. Student's t-test. *, p<0.05; * *, p<0.01; * * *, p<0.001. Data represent two independent experiments. FIG. 5B, Quantification of NK cells (NK1.1+/CD3−) isolated from the blood of tumor-bearing mice in the indicated treatment groups on day 24 as described in FIG. 4D. Mean±SEM, n=7. Student's t-test. *, p<0.05; **, p<0.01; * * *, p<0.001. FIG. 5C, Quantification of the percentage of iNK cell (CD27+CD11b−; top panel) and mNK cell (CD27−CD11b+; bottom panel) subpopulations in splenic NK cells isolated from tumor-bearing mice receiving the indicated treatments on day 24 as described in FIG. 4D. Mean±SEM, n=7. Student's t-test. *, p<0.05; **, p<0.01; * * *, p<0.001. FIG. 5D, Schematic of myeloid cell populations. FIG. 5E, Representative flow cytometry plots of the CD11b+ cell subpopulations in BM samples isolated from tumor-bearing mice receiving the indicated treatments as described in FIG. 4D. FIG. 5F, Quantification of the percentage of Ly6C^{high} Ly6G^{low} TAM precursor (left panel) and Ly6C^{low} Ly6G^{high} tumor-associated neutrophil (TAN) precursor (right panel) subpopulations in bone marrow (BM) CD11b+ cells isolated from mice on day 24 as described in FIG. 4D. Mean±SEM, n=7. Student's t-test. *, p<0.05; **, p<0.01; ***, p<0.001.

FIGS. 6A-6E. Neuroblastoma sEVs rescued the tumor growth which was attenuated by Tipifarnib and rescued the resistance to Dinutuximab which was suppressed by Tipifarnib. FIG. 6A, Experimental design. C57BL/6 mice were subcutaneously inoculated with 1×10⁶ 9464D-GD2 cells. One-week post-inoculation, tumor-bearing mice began receiving tail-vein injections of PBS control, dinutuximab or dinutuximab plus sEVs twice per week combined with oral administration of vehicle or tipifarnib twice per day. On day 24, tumors were harvested for analysis. FIG. 6B, Quantification of tumor volume at indicated time points. Mean±SEM. PBS, n=7; tipifarnib, n=11; tipifarnib+sEVs, n=10; dinutuximab, n=8; dinutuximab+tipifarnib, n=10; dinutuximab+tipifarnib+sEVs, n=10. FIG. 6C, Representative images of tumors from indicated treatment groups on day 24. Scale bar, 1 cm. FIG. 6D, Quantification of tumor weight from indicated treatment groups on day 24. Mean±SEM, n as in B. Student's t-test. *, p<0.05; **, p<0.01; * * *, p<0.001. FIG. 6E, Quantification of tumor-infiltrating NK cells (NK1.1+/CD3−; top panel) and tumor-associated macrophages (Mf) (CD11b+/F4/80+; middle panel) in 9464D-GD2 tumors isolated from mice in the indicated treatment groups. Mean±SEM. n=7 for PBS and Dinutuximab groups, n=9 for all other groups. Student's t-test. *, p<0.05; **, p<0.01; ***, p<0.001.

FIGS. 7A-7F. sEVs derived from parental 9464D cells induce comparable resistance to dinutuximab. FIG. 7A, Flow cytometry analysis of cell surface expression level of GD2 in 9464D cells and 9464D-GD2 cells. FIG. 7B, Schematic for sEV isolation from cell culture by differential ultracentrifugation. FIG. 7C, Flow cytometry analysis of GD2 expression on sEVs derived from 9464D cells and 9464D-GD2 cells absorbed onto latex beads (left). Median fluorescence intensity of GD2 corrected by unstained beads (right). Mean±SD, 9464D sEV, n=2; 9464D-GD2 sEV, n=3. Two-tailed unpaired t-test. **, p<0.01. FIG. 7D-FIG. 7F, C57BL/6 mice were subcutaneously inoculated with 1×10⁶ 9464D-GD2 cells. One-week post-inoculation, tumor-bearing mice began receiving tail-vein injections of PBS, sEVs derived from 9464D cells, dinutuximab (1.25 mg/kg) and sEVs derived from 9464D or 9464D-GD2 cells (20 μg) twice per week. On day 30, tumors were harvested for analysis. FIG. 7D, Quantification of tumor volume at the indicated time points. Mean±SEM, n=8 per group. FIG. 7E, Representative images of tumors at the end point. FIG. 7F, Tumor volume and weight at time of sacrifice. Mean±SEM, n=8 per group. Two-tailed unpaired t-test. *, p<0.05; **, p<0.01; ***, p<0.001.

FIGS. 8A-8F. sEVs modulate the transcriptome of immunoregulatory genes in 9464D-GD2 tumors while neuroblastoma patients with high expression of NK cell markers display a survival advantage. FIG. 8A, Experimental design for FIG. 8B-FIG. 8D and FIG. 2A-FIG. 2D. C57BL/6 mice were subcutaneously inoculated with 1×10⁶ 9464D-GD2 cells. One-week post-inoculation, tumor-bearing mice began receiving tail-vein injections with dinutuximab (1.25 mg/kg) or the combination of Dinutuximab (1.25 mg/kg) and sEVs (20 μg) twice per week. On day 23, tumors were harvested for RNA-sequencing analysis. FIG. 8B, Quantification of tumor volume at indicated time points. Mean±SEM, n=12 per treatment group. FIG. 8C, Quantification of tumor volume indicated treatment groups on day 23. Mean±SEM, n=12 per treatment group. Two-tailed unpaired t-test. *, p<0.05. FIG. 8D, Heatmap of differentially expressed genes for tumors treated with dinutuximab plus sEVs compared to dinutuximab alone. Vst transformed reads over row mean normalization. FIG. 8E, Kaplan-Meier Curve of event-free survival according to the expression of CD56 in neuroblastoma tumor. FIG. 8F, Kaplan-Meier Curve of event free survival according to the expression of NKG2D in neuroblastoma tumor. Data obtained from GSE62564 dataset.

FIGS. 9A-9D. Flow cytometry analysis gating strategies. FIG. 9A, Gating strategies for flow analysis of tumor-infiltrating NK cells and TAM. FIG. 9B, Gating strategies for flow analysis of blood NK cells. FIG. 9C, Gating strategies for flow analysis of splenic NK cells. FIG. 9D, Gating strategies for flow analysis of bone marrow CD11b+ cells.

FIGS. 10A-10B. Neuroblastoma-derived sEVs primarily localize to the liver, spleen, and lung in vivo. FIG. 10A, Whole-organ fluorescence was measured using the IVIS Imaging System at 6, 12 and 24 hours following tail vein injection of PBS or 30 g Vybrant DiD-labeled 9464D-GD2 sEVs into C57BL/6 mice. FIG. 10B, Quantification of whole-organ fluorescence intensities for lung, liver, spleen and heart. Mean±SEM, n=3. Student's t-test. *, p<0.05; **, p<0.01; ***, p<0.001.

FIGS. 11A-11D. Human neuroblastoma cells express GD2 on their surface and are sensitive to dinutuximab-induced NK cell-mediated ADCC that is suppressed by sEVs. FIG. 11A, Flow cytometry analysis of cell surface expression level of GD2 in EVIR32 human neuroblastoma cell line. FIG. 11B, Experimental design for the in vitro NK-cell-mediated ADCC assay. FIG. 11C, Representative images of IMR32 cells treated as indicated in the presence or absence of NK92-CD16-EGFP cells (NK cells) and IMR32-derived sEVs. Images were obtained using the IncuCyte Live-Cell Analysis System in the presence of the cell impermeant nucleic acid stain YOYO-3 (red). Scale bar, 400 μm. FIG. 11D, Kinetic analysis of the 9464D-GD2 in vitro NK-cell-mediated ADCC assay. Mean±SEM, n=4.

FIGS. 12A-12E. Loss of Rab27a inhibits 9464D tumor growth in vivo. FIG. 12A, Immunoblot of Rab27a in whole cell lysate of parental 9464D cells and 9464D crRab27a cells. All lanes loaded with equal amount of protein. FIG. 12B, Representative size distribution and concentration of sEVs isolated from 9464D cells and 9464D crRab27a cells quantified by NTA. FIG. 12C, Proliferation of 9464D cells and 9464D crRab27a cells quantified using the IncuCyte S3 Live-Cell Analysis System. Mean±SEM, n=6. FIG. 12D, Experimental design. C57BL/6 mice were subcutaneously inoculated with 1×10⁶ 9464D cells or 4×10⁶ 9464D crRab27a cells. On day 18, tumors were harvested for analysis. FIG. 12E, Quantification of 9464D tumor volume and 9464D crRab27a tumor volume on day 18. Mean±SEM, n=8. Student's t-test. **, p<0.01.

FIGS. 13A-13D. Tipifarnib inhibits sEV secretion in vitro and in vivo. FIG. 13A, Representative electron microscopy images of sEVs isolated from 9464D-GD2 cells treated with DMSO or 0.1 μM tipifarnib for 48 hours. Scale bar, 1 μm. FIG. 13B, Cell viability of 9464D cells at different concentrations of tipifarnib after 24, 48, and 72 hours. Mean±SD, n =6. FIG. 13C, NTA analysis of the size distribution and concentration of serum sEVs isolated from mice on day 24. Treatment as described in FIG. 4D. FIG. 13D, Quantification of the serum sEVs by NTA. Serum sEVs were isolated from mice on day 24 after receiving the indicated treatments as described in FIG. 4D. Mean±SEM, n=3. Student's t-test. *, p<0.05.

### DETAILED DESCRIPTION

Anti-GD2 monoclonal antibody immunotherapy has significantly improved the overall survival rate for high-risk neuroblastoma patients. However, 40% of patients fail to respond or develop resistance to the treatment, and the molecular mechanisms by which this occurs remain poorly understood. Here, the inventors utilize the syngeneic 9464D-GD2 mouse model to investigate the role of neuroblastoma-derived small extracellular vesicles (sEVs) in developing resistance to the anti-GD2 monoclonal antibody dinutuximab. Strikingly, neuroblastoma-derived sEVs significantly attenuated the efficacy of dinutuximab in vivo. Mechanistically, RNA-sequencing and flow cytometry analysis of whole tumors revealed that neuroblastoma-derived sEVs modulate immune cell tumor infiltration upon dinutuximab treatment to create an immunosuppressive tumor microenvironment that contains more tumor-associated macrophages (TAMs) and fewer tumor-infiltrating NK cells. In addition, neuroblastoma-derived sEVs suppressed splenic NK cell maturation in vivo and dinutuximab-induced NK cell-mediated antibody-dependent cellular cytotoxicity in vitro to provide additional mechanisms to dinutuximab resistance. Importantly, tipifarnib, a farnesyltransferase inhibitor that inhibits sEV secretion, drastically enhanced the efficacy of dinutuximab in vivo and reversed the immunosuppressive effects of neuroblastoma-derived sEVs. Notably, tipifarnib modulated immature myeloid cells in the bone marrow to disfavor the formation of CD11b+Ly6C(high)Ly6G(low) cells that are precursors for TAMs. Taken together, these findings uncover a novel mechanism by which neuroblastoma-derived sEVs modulate immunosuppression to promote resistance to dinutuximab and provide that tipifarnib-mediated inhibition of sEV secretion can be used as a treatment strategy to enhance the anti-tumor efficacy of anti-GD2 immunotherapy in high-risk neuroblastoma patients.

Herein, the inventors utilize a well-characterized pre-clinical mouse model of neuroblastoma to reveal that neuroblastoma-derived sEVs induce resistance to anti-GD2 immunotherapy. The inventors show that neuroblastoma-derived sEVs modulate the systemic immune response and alter immune cell tumor infiltration upon dinutuximab treatment to establish an immunosuppressive tumor microenvironment to evade dinutuximab-induced cytotoxicity. Importantly, the inventors identify tipifarnib, an FDA-approved farnesyltransferase inhibitor shown to inhibit sEV secretion, as a novel agent that enhances the efficacy of dinutuximab and reverses the immunosuppressive effects of neuroblastoma-derived sEVs. Taken together, the results provide a new treatment option that can be rapidly translated to the clinic to improve the outcome of high-risk neuroblastoma patients.

Disclosed herein are compositions comprising an anti-GD2 immunotherapy and a farnesyltransferase inhibitor, including those wherein the farnesyltransferase inhibitor comprises tipifarnib and/or the anti-GD2 immunotherapy comprises dinutuximab.

In some embodiments, the composition comprises a synergistic amount of a farnesyltransferase inhibitor and an anti-GD2 immunotherapy. In some embodiments, the composition comprises a synergistic amount of tipifarnib and dinutuximab.

Also disclosed are method of treating a cancer comprising administering to a subject in need thereof an anti-GD2 immunotherapy and a farnesyltransferase inhibitor, including those wherein the cancer is neuroblastoma or high-risk neuroblastoma.

Anti-GD2 immunotherapy treatment of neuroblastoma and can be grouped into first-generation and second-generation antibodies. First-generation anti-GD2 antibodies include: 14G2a; ch14.18; and 3F8. Second-Generation anti-GD2 antibodies include: Hu14.18-IL-2; Hu14.18K332A; and mAb1A7. All of these antibodies are going through clinical trial processes for the treatment of neuroblastoma. The most extensively studied of these antibodies is ch14.18. Matthay, Katherine K.; George, Rani E.; Yu, Alice K. (2012). “Promising therapeutic targets in neuroblastoma”. Clin Cancer Res. 18 (10): 2740-2753. doi:10.1158/1078-0432.ccr-11-1939. PMC 3382042. PMID 22589483.

In some embodiments, the farnesyltransferase inhibitor is selected from the group consisting of tipifarnib, lonafarnib (SCH-66336), CP-609,754, BMS-214662, L778123, L744823, L739749, R208176, AZD3409 and FTI-277. In some embodiments, the farnesyltransferase inhibitor is administered at a dose of 1-1000 mg/kg body weight.

In some embodiments, the anti-GD2 immunotherapy is dinutuximab and/or the farnesyltransferase inhibitor is tipifarnib. In one embodiment, the farnesyltransferase inhibitor is tipifarnib.

Further, methods herein include those wherein tipifarnib is administered according to at least one or more protocols selected from the group consisting of: at a dose of 1-1000 mg/kg body weight; once to twice a day; at a dose of 600 mg twice a day to 900 mg twice a day; dosed for a period of one to seven days.

In some embodiments, tipifarnib is administered at a dose of 200-1200 mg twice a day (“b.i.d.”). In some embodiments, tipifarnib is administered at a dose of 600 mg daily orally. In some embodiments, tipifarnib is administered at a dose of 300 mg b.i.d. orally for 3 of out of 4 weeks in repeated 4 week cycles. In some embodiments, tipifarnib is administered at a dose of 600 mg b.i.d. orally for 3 of out of 4 weeks in repeated 4 week cycles. In some embodiments, tipifarnib is administered at a dose of 900 mg b.i.d. orally in alternate weeks (one week on, one week off) in repeated 4 week cycles (days 1-7 and 15-21 of repeated 28-day cycles). In some embodiments, tipifarnib is administered at a dose of 1200 mg b.i.d. orally in alternate weeks (days 1-7 and 15-21 of repeated 28-day cycles). In some embodiments, tipifarnib is administered at a dose of 1200 mg b.i.d. orally for days 1-5 and 15-19 out of repeated 28-day cycles. In some embodiments, patients receive at least three cycles of treatment. In some embodiments, patients receive at least six cycles of treatment.

In other embodiments, the methods of treating a cancer further comprise administering a therapeutically effective amount of at least one or more active agents or a support care therapy selected from the group comprising: a DNA-hypomethylating agent, therapeutic antibody that specifically binds to a cancer antigen, a hematopoietic growth factor, a cytokine, an antibiotic, a cox-2 inhibitor, an immunomodulatory agent, an anti-thymocyte globulin, an immunosuppressive agent, and a corticosteroid.

In some embodiments, the administration of the therapeutically effective amount of the anti-GD2 immunotherapy and the farnesyltransferase inhibitor provides an increase in immune cell tumor infiltration, a decrease in tumor-associate macrophages, an increase in tumor-infiltrating natural killer (NK) cells, an increase in splenic NK cell maturation, an increase in NK cell-mediated antibody-dependent cellular cytotoxicity, a decrease of immunosuppressive effects of neuroblastoma-derived sEVs, or a decrease of formation of CD11b+Ly6C(high)Ly6G(low) cells.

In some embodiments, the subject comprises one or more cell markers corresponding to low expression of NKG2D and/or RAS/MAPK signal pathway deficiencies.

In some embodiments, the neuroblastoma is a GD2 inhibitor-refractory neuroblastoma (GIRN).

Also disclosed herein are methods of preventing neuroblastoma metastasis, comprising administering to a subject a therapeutically effective amount of an anti-GD2 immunotherapy and a farnesyltransferase inhibitor, including those wherein the anti-GD2 immunotherapy is dinutuximab and/or wherein the farnesyltransferase inhibitor is tipifarnib.

In some embodiments, the method of preventing neuroblastoma metastasis further comprises administering a therapeutically effective amount of at least one or more active agents or a support care therapy selected from the group consisting of a DNA-hypomethylating agent, therapeutic antibody that specifically binds to a cancer antigen, a hematopoietic growth factor, a cytokine, an antibiotic, a cox-2 inhibitor, an immunomodulatory agent, an anti-thymocyte globulin, an immunosuppressive agent, and a corticosteroid.

In other embodiments, the method of preventing neuroblastoma metastasis further comprises administration of the therapeutically effective amount of the anti-GD2 immunotherapy and the farnesyltransferase inhibitor provides an increase in immune cell tumor infiltration, a decrease in tumor-associate macrophages, an increase in tumor-infiltrating natural killer (NK) cells, an increase in splenic NK cell maturation, an increase in NK cell-mediated antibody-dependent cellular cytotoxicity, a decrease of immunosuppressive effects of neuroblastoma-derived sEVs, or a decrease of formation of CD11b+Ly6C(high)Ly6G(low) cells.

Also provided are methods of preventing neuroblastoma metastasis, comprising: (a) determining the presence or absence of low-expression cell markers for NKGD2 in a sample from said subject, and subsequently (b) administering a therapeutically effective amount of an anti-GD2 immunotherapy and a farnesyltransferase inhibitor to said subject if the sample is determined to have low expression cell markers for NKGD2.

In some embodiments, the methods provided herein also include administering additional therapies to the subject. The additional therapy can be a radiation therapy. In some embodiments, the methods provided herein also include administering a therapeutically effective amount of an additional active agent or a support care therapy to the subject. In some embodiments, the additional active agent is a DNA-hypomethylating agent, a therapeutic antibody that specifically binds to a cancer antigen, a hematopoietic growth factor, cytokine, anti-cancer agent, antibiotic, cox-2 inhibitor, immunomodulatory agent, anti-thymocyte globulin, immunosuppressive agent, corticosteroid or a pharmacologically derivative thereof. In some embodiments, the secondary active agent is a DNA-hypomethylating agent, such as azacitidine or decitabine.

Included are methods of treating a GD2 inhibitor-refractory neuroblastoma (GIRN) in a subject having cell markers corresponding to low expression of NKG2D and/or RAS/MAPK signal pathway deficiencies, comprising administering a therapeutically effective amount of an anti-GD2 immunotherapy and a farnesyltransferase inhibitor to the subject. Also included are methods of treating neuroblastoma in a subject, comprising: (a) obtaining a sample from the subject; (b) determining presence or absence of low-expression cell markers for NKGD2 and/or RAS/MAPK signal pathway deficiencies in the sample from said subject, and subsequently (c) administering a therapeutically effective amount of an anti-GD2 immunotherapy and a farnesyltransferase inhibitor to said subject if the sample is determined to have low expression cell markers for NKGD2 and/or RAS/MAPK signal pathway deficiencies. Other methods of preventing neuroblastoma metastasis are provided, comprising administering a therapeutically effective amount of an anti-GD2 immunotherapy and a farnesyltransferase inhibitor to said subject.

In addition, disclosed are tipifarnib and dinutuximab in combination for the manufacture of a medicament for the use in the treatment of neuroblastoma, as are tipifarnib and dinutuximab in combination for the manufacture of a medicament for the use in the treatment of dinutuximab-resistant high risk neuroblastoma, and tipifarnib and dinutuximab in combination for the manufacture of a medicament for the use in the treatment of neuroblastoma having low expression cell markers for NKGD2 and/or RAS/MAPK signal pathway deficiencies.

In some embodiments, the method comprises administering to a subject a synergistic amount of a farnesyltransferase inhibitor and an anti-GD2 immunotherapy. In some embodiments, the method comprises administering to a subject a synergistic amount of a tipifarnib and dinutuximab.

### Terminology

Terms used throughout this application are to be construed with ordinary and typical meaning to those of ordinary skill in the art. However, Applicant desires that the following terms be given the definition as defined below.

As used in the specification and claims, the singular form “a,” “an,” and “the” include plural references unless the context clearly dictates otherwise. For example, the term “a particle” includes a plurality of particles, including mixtures thereof.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 10% (e.g., within 2% or 1%) of the particular value modified by the term “about.”

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22: 1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988).

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

In some aspects, disclosed herein are methods of preventing, reducing, inhibiting, and/or treating diseases, including sEV-impacted pathologies, neuroblastoma, and high-risk neuroblastoma, comprising administering to the subject in need a therapeutically effective amount of the compositions disclosed herein.

In some aspects, disclosed herein are method of preventing, reducing, inhibiting, and/or treating diseases, including sEV-impacted pathologies, including neuroblastoma and high-risk neuroblastoma, comprising administering to the subject in need a therapeutically effective amount of a composition disclosed herein.

In some embodiments, the compositions described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavemous, intrathecal, intravitreal, intracerebral, gingival, subgingival, intracerebroventricular, and intradermal. Such formulations may be prepared by any method known in the art.

The disclosed methods can be performed any time prior to and/or after the onset of disease. In some aspects, the disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the onset of disease; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48.60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the onset of disease.

Dosing frequency disclosed herein includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiment, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiment, the dosing frequency disclosed herein includes, but is not limited to, at least once a day, twice a day, or three times a day. In some embodiment, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiment, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiment, the interval between each administration is constant. For example, the administration can be carried out daily, every two days, every three days, every four days, every five days, or weekly. Administration can also be continuous and adjusted to maintaining a level of the compound within any desired and specified range.

The methods provided are useful in researching, treating, reducing, decreasing, inhibiting, and/or preventing sEV-impacted pathologies, including neuroblastoma and high-risk neuroblastoma.

Those skilled in the art will recognize or be able to ascertain using no more than routine experimentation, many equivalents to the specific embodiments of the invention described herein. While the invention has been described with reference to particular embodiments and implementations, it will be understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention is not limited to the particular implementations disclosed herein, but that the invention will include all implementations falling within the scope of the appended claims.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1. Small Extracellular Vesicles Induce Resistance to Anti-GD2 Immunotherapy

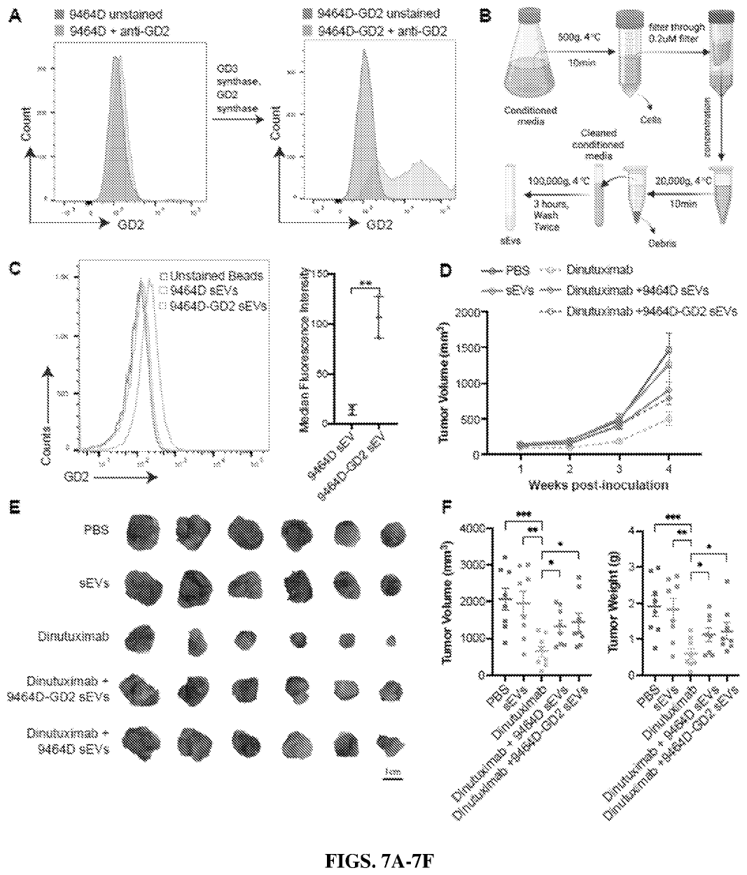

Neuroblastoma-Derived sEVs Induce Resistance to Dinutuximab In Vivo The 9464D cell line is derived from a spontaneous neuroblastoma arising in a TH-MYCN transgenic mouse on the C57BL/6 background to provide a genetically-defined and transplantable immunocompetent model of neuroblastoma (21,22). While the complex, acidic glycolipid GD2 is highly expressed in most neuroblastoma, 9464D cells have been reported to express a lower level of cell surface GD2 compared to other neuroblastoma cell lines (22,23). To establish a syngeneic model of neuroblastoma suitable for investigating mechanisms of resistance to anti-GD2 immunotherapy, murine GD3 synthase (St8sia1), the rate-limiting enzyme for GD2 biosynthesis, and GM2/GD2 synthase (B4galnt1) were stably overexpressed in 9464D cells to upregulate GD2 expression on the cell surface (9464D-GD2; FIG. 7A).

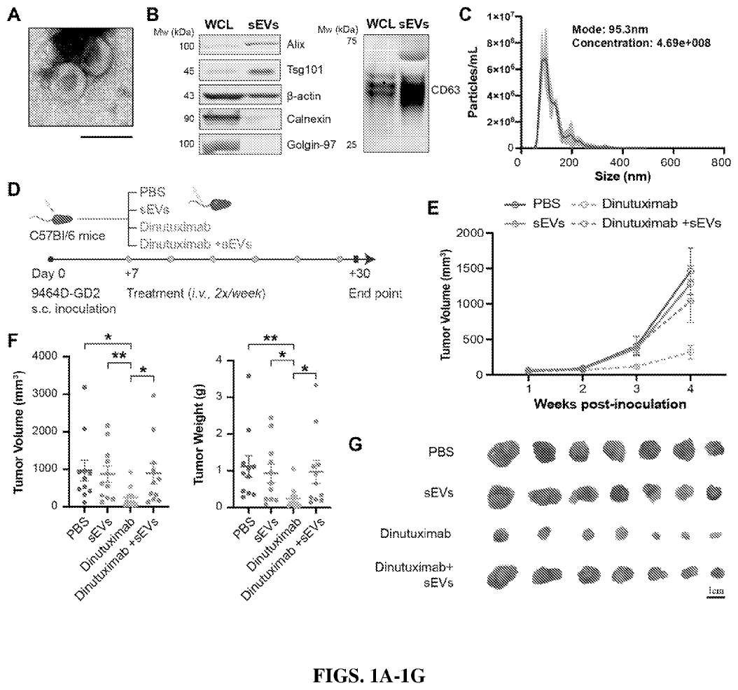

sEVs were isolated from 9464D-GD2 cells using a well-established differential ultracentrifugation protocol (19) (FIG. 7B) and were found to demonstrate the characteristic morphology, protein markers and size distribution of sEVs (FIG. 1A-C) (24). To determine how sEVs regulate tumor growth and resistance to anti-GD2 immunotherapy in vivo, immunocompetent C57BL/6 mice were subcutaneously inoculated with 1×10⁶ 9464D-GD2 cells. One-week post-inoculation, tumor-bearing mice began receiving twice-weekly tail-vein injections of PBS, dinutuximab, and/or sEVs isolated from 9464D-GD2 cells (hereafter denoted sEVs unless otherwise noted) and monitored for tumor growth (FIG. 1D). Dinutuximab dramatically suppressed tumor growth compared to PBS control to highlight the efficacy of anti-GD2 immunotherapy against 9464D-GD2 tumors in vivo (FIG. 1E). Strikingly, while sEVs alone failed to alter tumor growth compared to control, mice treated with dinutuximab and sEVs displayed nearly complete resistance to the anti-tumor effects of dinutuximab (FIG. 1E). Ex vivo quantification of tumor volume and weight at the experimental endpoint confirmed that sEVs significantly suppressed the anti-tumor efficacy of dinutuximab while having no effect on 9464D-GD2 tumor growth when administered alone (FIGS. 1F and G).

While the cell surface expression of GD2 is significantly upregulated in 9464D-GD2 cells, sEVs isolated from these cells only showed a mild increase in GD2 levels (FIG. 7C). However, to investigate whether GD2-containing sEVs are an important contributor to dinutuximab resistance, the inventors repeated the in vivo experiment and included sEVs derived from 9464D-GD2 and parental 9464D cell lines (FIG. 7D). Notably, sEVs derived from both cell lines suppressed the anti-tumor efficacy of dinutuximab to a comparable extent (FIG. 7D-F), providing that sEV-associated GD2 is not a primary factor involved in mediating resistance to anti-GD2 immunotherapy in this model.

Neuroblastoma-Derived sEVs Inhibit Dinutuximab-Induced NK Cell Tumor Infiltration and Enhance the Recruitment of Tumor Associated Macrophages

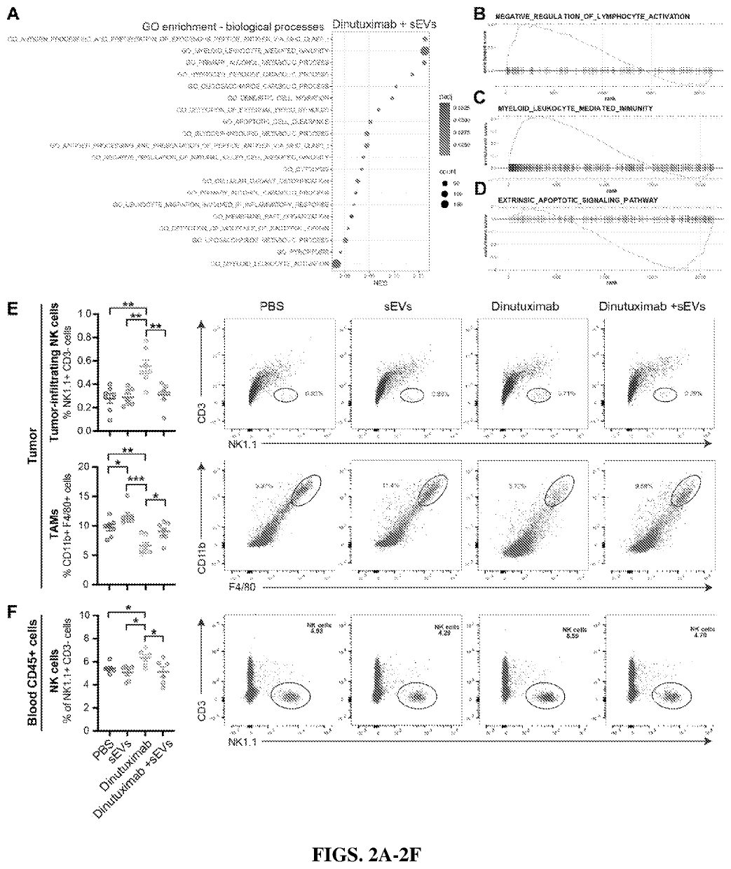

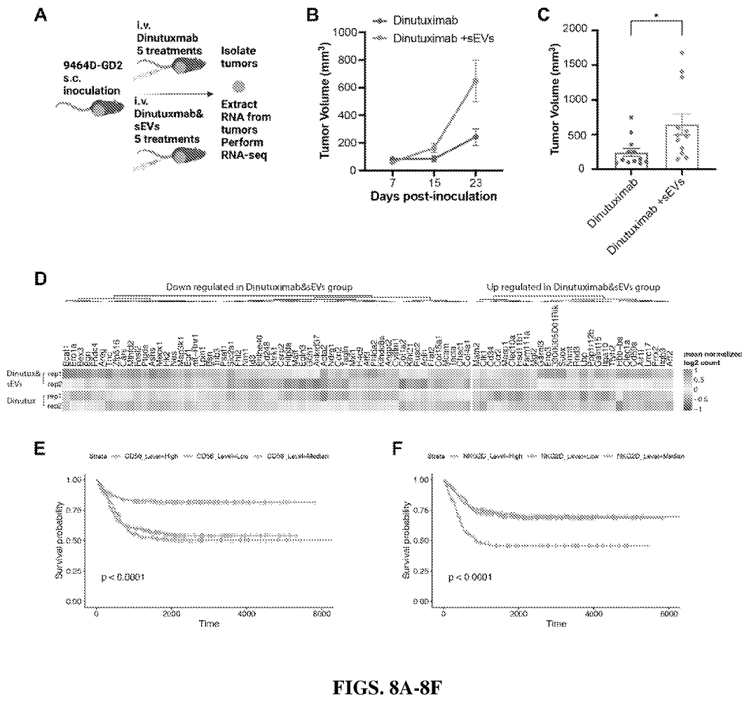

To understand the transcriptome changes responsible for sEV-mediated resistance to dinutuximab, the inventors performed RNA sequencing analysis of 9464D-GD2 tumors isolated from C57BL/6 mice treated with dinutuximab or dinutuximab plus sEVs (FIG. 8A-C). Interestingly, tumors derived from mice treated with the combination of dinutuximab and sEVs demonstrated a significant upregulation in genes involved in myeloid leukocyte-mediated immunity and myeloid cell recruitment, including Ccr2 (25), Clec10a (26), and Clec1a (27) (FIG. 8D). To further investigate how sEVs mediate changes to the tumor microenvironment (TME), the inventors performed Gene Set Enrichment Analysis (GSEA). Intriguingly, pathways involved in myeloid leukocyte-mediated immunity and the negative regulation of NK cell-mediated immunity and cytotoxicity were among the top 20 most significantly enriched pathways in tumors derived from mice treated with dinutuximab plus sEVs (FIG. 2A).

Using GSEA of gene sets of interest, the inventors confirmed that sEVs induced an enrichment of genes involved in myeloid leukocyte-mediated immunity as well as the negative regulation of lymphocyte activation (FIGS. 2B and C). In contrast, genes involved in extrinsic apoptotic signaling were less enriched in tumors treated with dinutuximab and sEVs compared to dinutuximab alone (FIG. 2D).

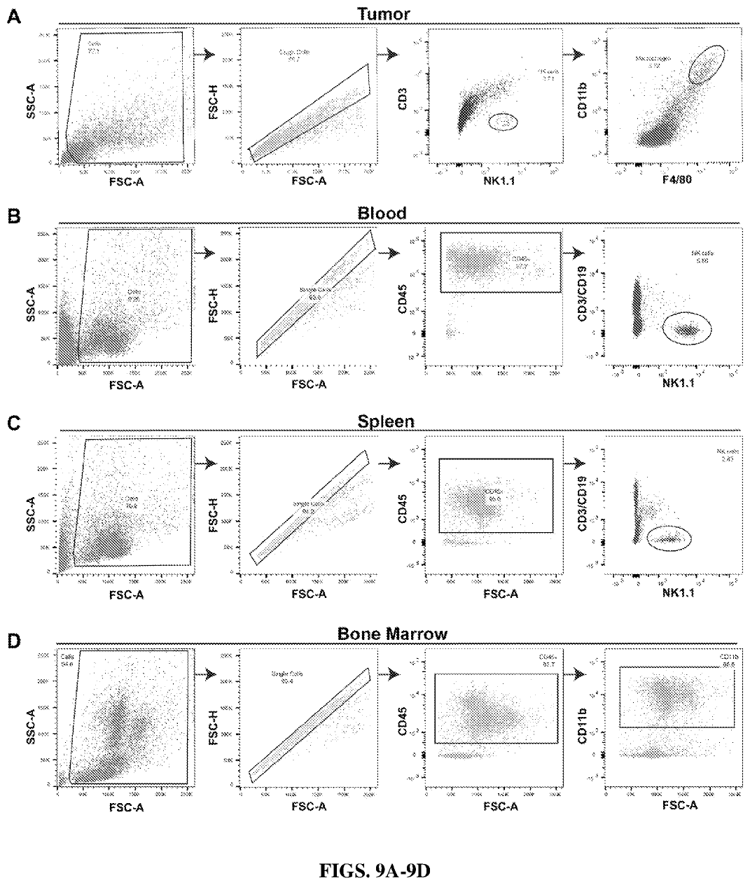

As the tumor-infiltrating immune cell composition is highly correlated to the sensitivity to immunotherapy, the inventors hypothesized that neuroblastoma-derived sEVs alter the tumor immune microenvironment. To test this hypothesis, the inventors performed flow cytometry analysis for immune cell populations in tumors derived from mice treated with PBS, dinutuximab and/or sEVs (as in FIG. 1). The inventors were specifically interested in how NK cell tumor infiltration was affected by sEVs, as patients with tumors that express high levels of the NK cell marker CD56 or NK cell activating receptor NKG2D demonstrate an increased event-free 5-year survival probability compared to patients with low tumor expression of these markers (FIG. 8E-F). The inventors found that dinutuximab significantly increased the tumor-infiltrating NK cell population compared to PBS control or sEVs alone (FIG. 2E, top panel; see FIG. 9A for gating strategy). Additionally, mice treated with dinutuximab displayed a significant increase in the percentage of serum NK cells compared to PBS and sEVs treatment groups (FIG. 2F; see FIG. 9A for gating strategy) to show that dinutuximab mobilizes NK cells to the primary tumor site. Strikingly, sEVs significantly suppressed dinutuximab-induced NK cell mobilization and tumor infiltration to a level comparable to control (FIG. 2E, top panel and F).

Myeloid cells also play important roles in regulating the response of neuroblastoma to immunotherapy, as they create an immunosuppressive TME that suppresses the proliferation and cytotoxicity of T cells and NK cells (3). As the RNA sequencing results implicated myeloid cells in sEV-mediated resistance to dinutuximab, the inventors next examined the presence of tumor-associated macrophages (TAMs). Dinutuximab significantly decreased the TAM population compared to all other treatment groups (FIG. 2E, bottom panel). Interestingly, sEVs significantly upregulated the percentage of TAMs compared to control when administered alone and restored the TAM population to control-like levels when combined with dinutuximab (FIG. 2E, bottom panel). Collectively, these results show that neuroblastoma-derived sEVs induce resistance to dinutuximab by promoting an immunosuppressive TME characterized by decreased tumor-infiltrating NK cells and increased TAM.

Neuroblastoma-Derived sEVs Modulate NK Cell Maturation In Vivo and NK Cell-Mediated ADCC In Vitro

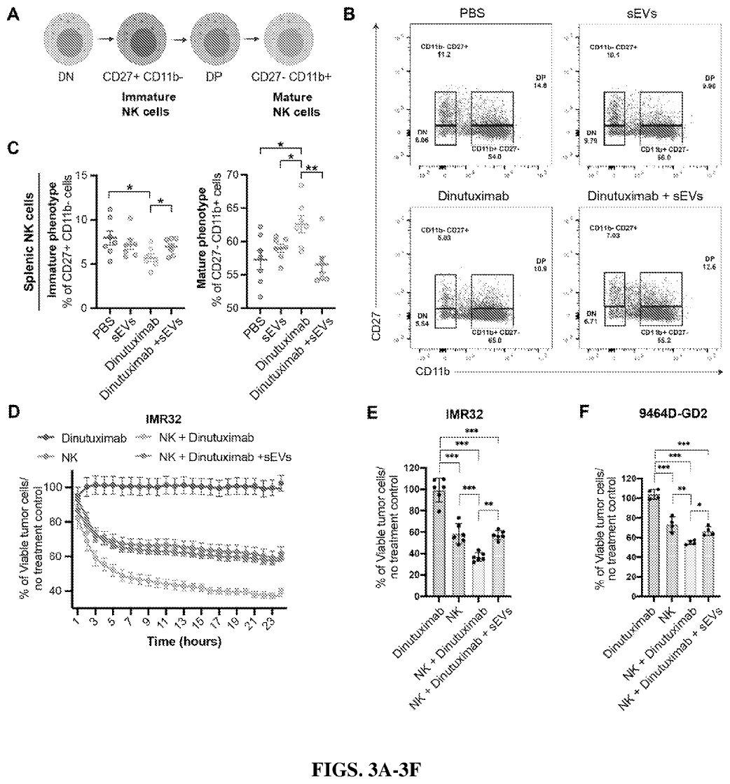

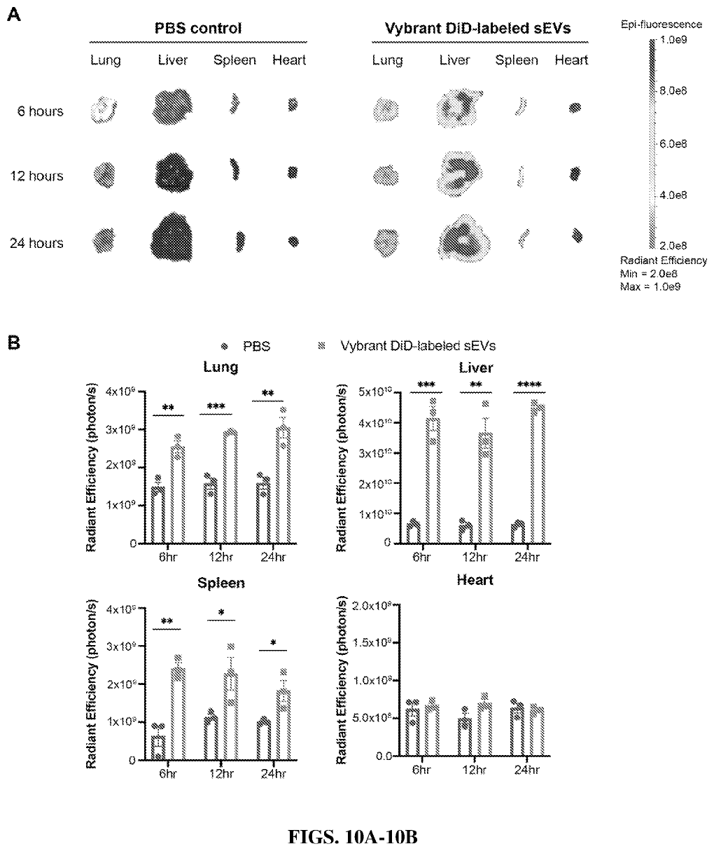

The inventors found that neuroblastoma-derived sEVs labeled with Vybrant DiD and injected into the tail vein of mice were primarily taken up by the liver, spleen and lung (FIG. 10). As splenic uptake of neuroblastoma-derived sEVs may be capable of modulating immune cell maturation, the inventors next asked whether neuroblastoma-derived sEVs alter the systemic immune response to promote resistance to anti-GD2 immunotherapy. To this end, the inventors performed flow cytometry analysis on spleens isolated from 9464D-GD2 tumor-bearing mice treated with PBS, dinutuximab and/or sEVs as in FIG. 1. Since NK cells are the main immune effector for responding to anti-GD2 immunotherapy, the inventors were particularly interested in how neuroblastoma sEVs modulate NK cell subpopulations and maturation in the spleen (3). The inventors used CD27 and CD11b to subdivide NK cells into four different maturation subsets and focused on CD27+CD11b−cells displaying an immature phenotype and CD27−CD11b+ cells exhibiting a mature phenotype (FIGS. 3A and B) (29,30). Interestingly, dinutuximab significantly decreased the percentage of splenic NK cells with an immature phenotype and increased the percentage of mature splenic NK cells compared to all other treatment groups to demonstrate that dinutuximab promotes splenic NK cell maturation in vivo (FIG. 3C). Notably, the combination of dinutuximab and sEVs restored the splenic NK cell subsets to control levels to show that neuroblastoma-derived sEVs suppress dinutuximab-induced NK cell maturation in vivo (FIG. 3C).

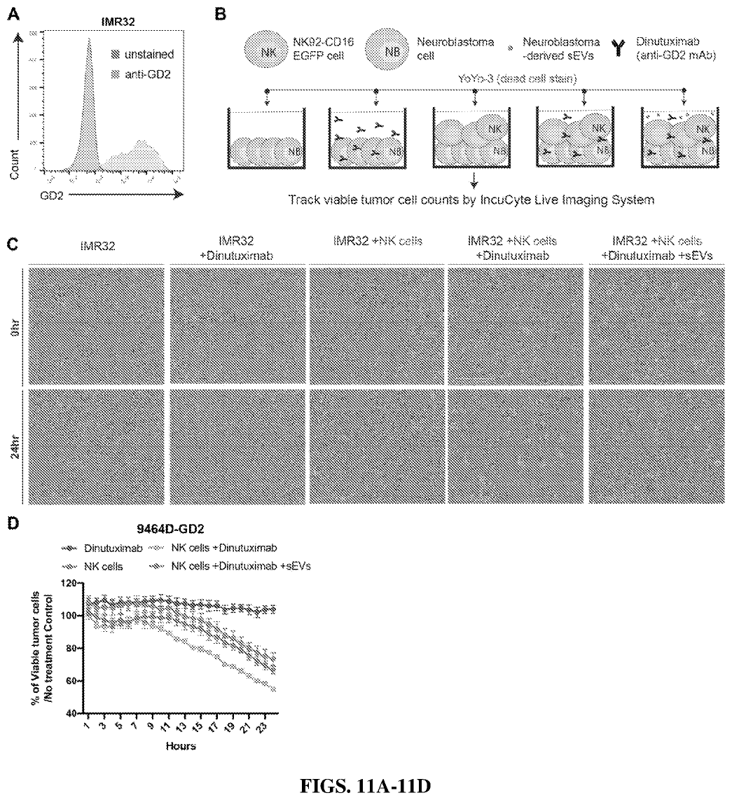

The inventors then asked whether neuroblastoma-derived sEVs directly suppress NK cell-mediated ADCC by performing an in vitro co-culture experiment using the NK92 cell line stably expressing CD16 and EGFP (NK92-CD16-EGFP) and either 9464D-GD2 or the human GD2-positive neuroblastoma cell line EVIR32 (FIGS. 11A and B). NK cell-mediated ADCC was monitored in the presence or absence of dinutuximab and sEVs using the cell impermeant nucleic acid stain YOYO-3 and the IncuCyte S3 Live-Cell Analysis System. The cytotoxic effects of dinutuximab against human and murine neuroblastoma cells required the presence of NK92-CD16-EGFP immune effector cells (FIG. 3D-F; FIG. 11C-E), indicating that dinutuximab initiates tumor cell killing through activation of NK cell-mediated ADCC. Pre-incubation of NK92-CD16-EGFP cells with neuroblastoma-derived sEVs suppressed dinutuximab-induced NK cell-mediated ADCC against neuroblastoma cell lines (FIG. 3D-F; FIG. 11C-D). Taken together, these data show that neuroblastoma-derived sEVs induce resistance to dinutuximab by both modulating NK cell maturation in vivo and suppressing NK cell-mediated ADCC.

Inhibition of Small Extracellular Vesicle (sEV) Secretion by Tipifarnib Sensitizes 9464D-GD2 Tumors to Dinutuximab

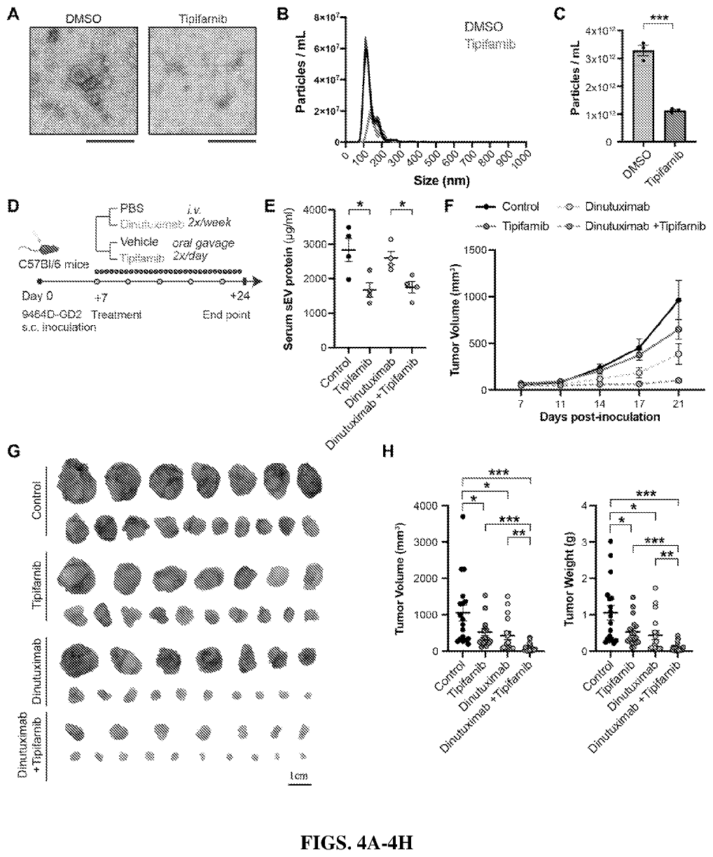

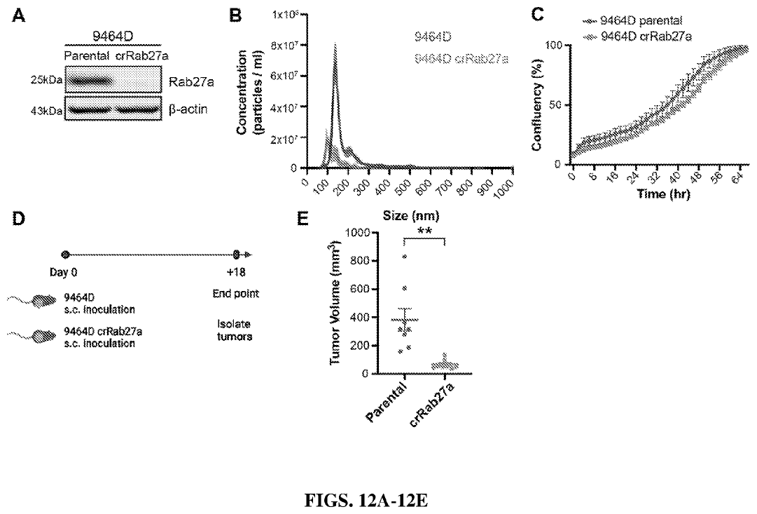

After determining that neuroblastoma-derived sEVs induce resistance to anti-GD2 immunotherapy through modulation of the immune system, the inventors sought to determine whether inhibition of sEV secretion would re-sensitize the tumor to dinutuximab. To this end, the inventors attempted to block sEV secretion by genetic depletion of Rab27a, an essential Rab GTPase involved in sEV secretion (24). Loss of Rab27a in 9464D cells dramatically suppressed sEV secretion in vitro (9464D-crRab27a, FIG. 12A-C). However, despite showing no difference in proliferation in vitro, loss of Rab27a dramatically suppressed 9464D tumor growth in C57BL/6 mice (FIG. 12E). While this result is agreement with previous reports in other cancer models (13,31), it precluded using the genetic model to dissect the role of neuroblastoma-derived sEVs in dinutuximab resistance. As an alternative approach, the inventors sought to inhibit sEV secretion in vivo using tipifarnib. Tipifarnib is a potent, selective and orally bioavailable inhibitor of farnesyltransferase that has recently been shown to selectively inhibit sEV secretion from cancer cell lines in vitro (32). As tipifarnib inhibits additional cellular pathways including RAS signaling, the inventors demonstrated that a dose of 0.1 μM tipifarnib was able to significantly suppress sEV secretion in 9464D-GD2 cells in the absence of cytotoxicity (FIG. 4A-C, FIG. 12A-B).

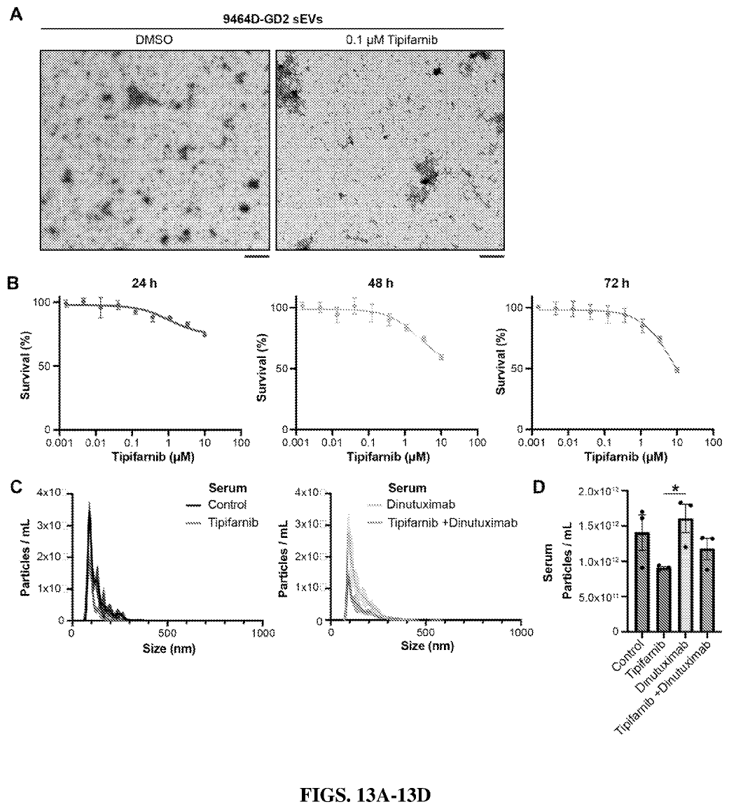

To determine whether tipifarnib sensitizes neuroblastoma tumors to dinutuximab in vivo, the inventors subcutaneously inoculated C57BL/6 mice with 1×10⁶ 9464D-GD2 cells. One-week post-inoculation, tumor-bearing mice began receiving twice-weekly tail-vein injections of PBS or dinutuximab in combination with tipifarnib or an equivalent volume of vehicle by oral gavage two times per day (FIG. 4D). Mice treated with tipifarnib demonstrated a significant decrease in sEV secretion as determined by the measurement of serum sEV protein, confirming that tipifarnib suppresses sEV secretion in vivo (FIG. 4E). Moreover, NTA analysis demonstrated a reduction in circulating sEV concentration in mice treated with tipifarnib compared to control (FIGS. 13C and D). Interestingly, mice treated with tipifarnib alone showed a significant reduction in tumor volume and weight compared to vehicle to show that tipifarnib suppresses tumor growth in vivo through inhibition of sEV secretion and/or other cellular mechanisms (FIG. 4F-H). Notably, however, tipifarnib enhanced the anti-tumor efficacy of dinutuximab to significantly decrease tumor growth and size compared to all other treatment groups (FIG. 4F-H).

Tipifarnib Cooperates with Dinutuximab to Remodel the TME and Reverse the Systemic Immune Suppression Induced by Neuroblastoma-Derived sEVs

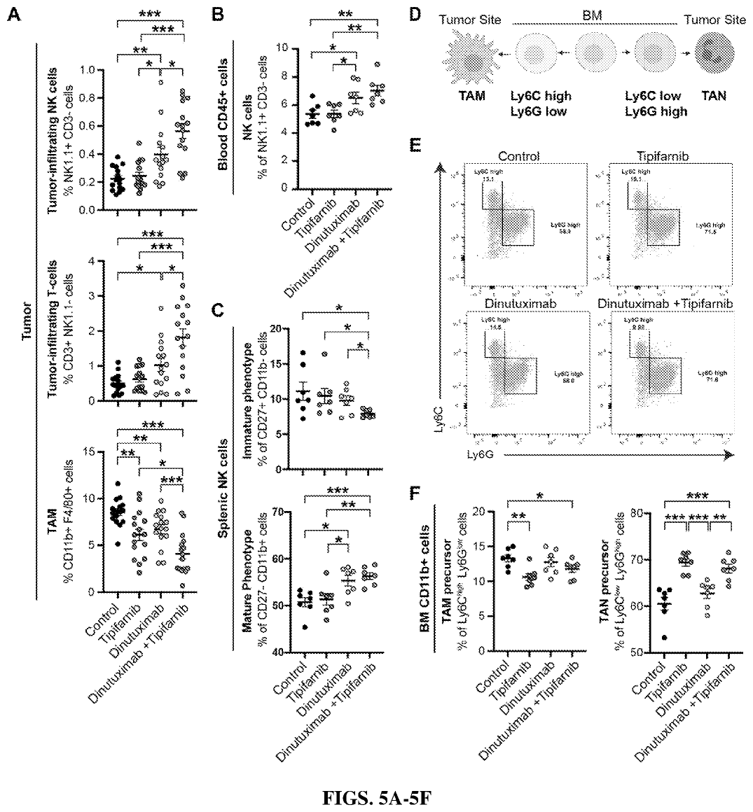

To examine whether tipifarnib sensitizes tumors to dinutuximab by reversing the immunosuppressive effects induced by neuroblastoma-derived sEVs, the inventors performed flow cytometry analysis of 9464D-GD2 tumors and blood isolated from mice treated with vehicle, dinutuximab and/or tipifarnib as in FIG. 4D. Consistent with the data above, dinutuximab promoted an immunoreactive TME by enhancing the mobilization of NK cells and percentages of tumor-infiltrating NK cells and T-cells while decreasing TAMs (FIGS. 5A and B). Tipifarnib strongly boosted the effects of dinutuximab by further enhancing the level of circulating NK cells and the recruitment of tumor-infiltrating NK cells and T-cells (FIGS. 5A and B) to show that tipifarnib and dinutuximab work synergistically to reverse the immunosuppressive TME. Interestingly, while sEVs enhanced the presence of TAMs (FIG. 2E), tipifarnib treatment alone significantly suppressed the TAM population, which was further suppressed in combination with dinutuximab to show that tumor-derived sEVs promote the recruitment of immunosuppressive TAMs (FIG. 5A).

To determine whether tipifarnib reverses the systemic immune suppression induced by neuroblastoma sEVs, the inventors examined NK cell subsets in the spleen and myeloid cell subpopulations in the bone marrow (BM). The inventors found that both dinutuximab and the combination of dinutuximab and tipifarnib significantly increased the percentage of mature NK cells in the spleen compared to control or tipifarnib alone (FIG. 5C). Moreover, the combination treatment decreased the percentage of splenic immature NK cells compared to other treatment groups (FIG. 5C). Recent literature revealed that TAMs are primarily derived from BM rather than splenic progenitor cells (33) and arise from the CD11b+Ly6C^{high} population of circulating mouse monocytes in grafted tumors (34). As tipifarnib altered the percentage of TAMs, the inventors next investigated whether tipifarnib alters myeloid cell populations in the BM where immature myeloid cells comprise a spectrum between monocytes and neutrophils (35). Cells that differentiate toward TAMs in solid tumors are CD11b+Ly6C^{high}Ly6G^{low}, while the cells that differentiate toward tumor-associated neutrophils (TANs) are CD11b+Ly6C^{low}Ly6G^{high} (FIG. 5D) (35). Tipifarnib significantly decreased the percentage of the CD11b+Ly6C^{high}Ly6G^{low} myeloid cells and increased the percentage of CD11b+Ly6C^{low}Ly6G^{high} myeloid cells in both the presence and absence of dinutuximab (FIGS. 5E and F, gating strategy in FIG. 9D).

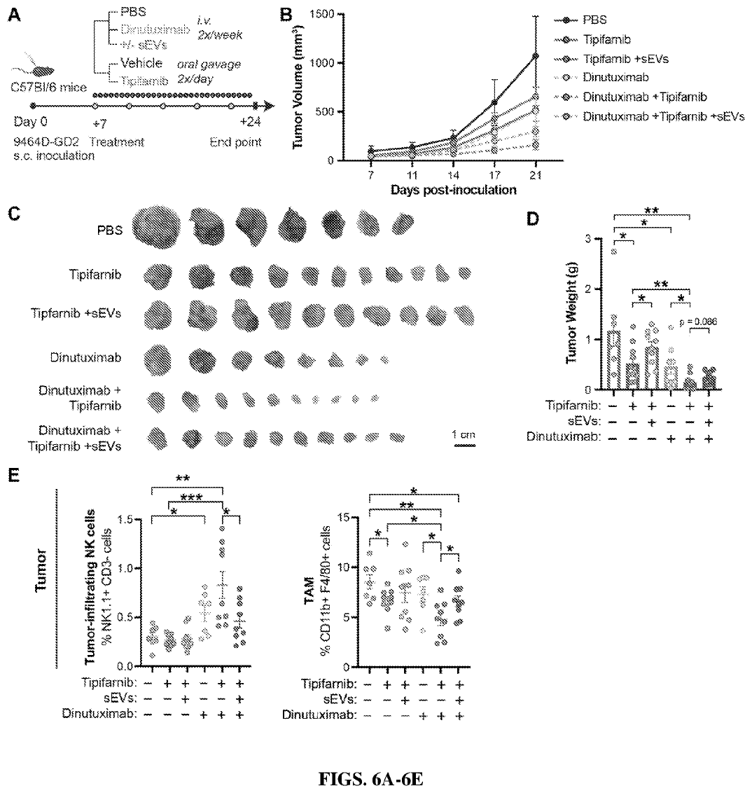

To validate that tipifarnib sensitizes neuroblastoma tumors to dinutuximab in vivo through the inhibition of sEV secretion, the inventors performed a rescue experiment in which neuroblastoma-derived sEVs were added to dinutuximab and/or tipifarnib treatments (FIG. 6A). Consistently, while dinutuximab and tipifarnib demonstrated mild anti-tumor activity alone, the combination of dinutuximab and tipifarnib significantly decreased tumor growth and weight compared to either agent alone (FIG. 6B-D). Notably, sEVs rescued tumor growth and weight of tipifarnib treated mice to a level comparable to control to suggest that inhibition of tumor-derived sEV secretion contributes to the anti-tumor effects of tipifarnib in vivo (FIG. 6B-D). Furthermore, sEVs appeared to reverse the anti-tumor efficacy of dinutuximab plus tipifarnib (FIG. 6B-D), albeit not statistically significant (p=0.086). Interestingly, the inventors found that the addition of sEVs to dinutuximab and tipifarnib treatment significantly decreased the presence of tumor infiltrating NK cells and enhanced the TAM population to a level comparable to dinutuximab treatment alone (FIG. 6E). Taken together, these results show that tipifarnib enhances the efficacy of anti-GD2 immunotherapy at least in part through inhibiting sEV secretion from neuroblastoma tumor.

### DISCUSSION

The inventors demonstrated that neuroblastoma-derived sEVs significantly suppressed the efficacy of dinutuximab in vivo and unveiled the FDA-approved drug tipifarnib as a promising novel adjunct to anti-GD2 immunotherapy. Markedly, sEV-mediated resistance to dinutuximab was independent of sEV-associated GD2 expression to establish that neuroblastoma-derived sEVs do not serve as an antibody decoy that inhibits the binding of tumor-reactive antibodies and tumor cells. Rather the data revealed that neuroblastoma-derived sEVs modulated immune effector cells both locally within the TME and systemically in the spleen. NK cells are the primary immune effector cells that mediate dinutuximab-induced killing.

Systemically, the inventors found that tumor-derived sEVs suppressed dinutuximab-induced NK cell maturation in the spleen and NK cell mobilization. Namely, sEVs suppressed the dinutuximab-induced infiltration of NK cells into tumors while enhancing the population of TAMs to favor an immunosuppressive TME. In agreement with the observations, intratumoral NK cells in human neuroblastoma predict improved overall survival while TAMs are associated with metastatic tumors and worse patient outcome Collectively, the data establish that neuroblastoma-derived sEVs modulate the tumor immune cell environment to confer resistance to dinutuximab.

The 9464D subcutaneous tumor model offers an immune microenvironment that is comparable to human neuroblastoma and provides a practical model for testing clinically applicable therapies. Dinutuximab is less efficient for targeting neuroblastoma cells in a solid tumor mass than neuroblastoma cells in the BM.

Tumor-derived sEVs are reported to have a complex role in modulating the response to immunotherapies. More specifically, tumor-derived sEVs carry a wide array of immunosuppressive cargo molecules, including miRNA, long non-coding RNA, DNA and proteins that interfere with the host immune system and reprogram immune effector cells. For example, melanoma-derived sEVs contain programmed death-ligand 1 (PD-L1), which interacts with programmed death-1 (PD-1) receptor on the surface of CD8-positive T cells to suppress T cell function and promote tumor growth. Moreover, tumor-derived sEVs have been shown to inhibit NK cell function by the transfer of miR-23a leading to the downregulation of CD107a. Similarly, tumor-derived EVs have been reported to carry transforming growth factor beta (TGF-β), which inhibits NK cell cytotoxicity by downregulating the expression of the activating receptor NKG2G.

The inventors demonstrated for the first time that tipifarnib significantly enhanced the anti-tumor efficacy of dinutuximab. Tipifarnib was recently identified in a high-throughput screen as a selective inhibitor of sEV secretion from cancer cells. Mechanistically, tipifarnib inhibited sEV secretion by downregulating several molecules involved in sEV biogenesis and/or secretion, including ALG-2-interacting protein X (Alix), neutral sphingomyelinase 2 and Rab27a. Tipifarnib is a potent farnesyltransferase inhibitor that has been shown to have anti-tumor activity by inhibiting pro-tumorigenic HRAS signaling. The data revealed that when given as a single agent tipifarnib exhibited mild anti-tumor efficacy in neuroblastoma that could be rescued by neuroblastoma-derived sEVs, indicating that inhibition of tumor-derived sEV secretion contributes to the anti-tumor efficacy of tipifarnib in this model. Likewise, neuroblastoma-derived sEVs partially rescued tumor growth and reversed the effects of tipifarnib and dinutuximab on tumor immune cell infiltration. Interestingly, anaplastic lymphoma kinase (ALK)-RAS/MAPK pathway alterations strongly correlate with poor outcome in all neuroblastoma risk categories and are found to be present at a higher frequency in relapsed neuroblastoma tumors. Therefore, in addition to inhibiting sEV secretion, tipifarnib administered with dinutuximab provides additional therapeutic benefits to patients harboring aberrant RAS/MAPK signaling pathways.

### Transduction and CRISPR Cas9-Mediated Genome Editing

The lentiCas9-Blast (#52962) and LRG (Lenti_sgRNA_EFS_GFP, #65656) plasmids were obtained from Feng Zhang and Christopher Vakoc, respectively, through Addgene. To produce recombinant lentiviruses, each lentiviral vector was co-transfected with the third-generation lentiviral packaging plasmids (pLP1, pLP2, and pLP/VSVG) into HEK 293T/17 cells using jetPRIMIE transfection reagent (Polyplus, 89129-922). Virus-containing supernatants were collected 24 and 48 hours after transfection, centrifuged (500 RCF) and filtered (0.45-am pore) prior to storage at −80° C. 9464D cells stably expressing Cas9 were generated by transducing 9464D cells with lentiCas9-Blast followed by selection with blasticidin (5 μg/mL). Cas9-expressing 9464D cells were transiently transfected with LRG constructs containing sgRNAs targeting murine Rab27a (5′-TGGTTAAGCTACGAAACCTA-3′ (SEQ ID NO: 3), 5′-AGTGTACTGGTAGAGTACAC-3′ (SEQ ID NO: 4), 5′-AACCCAGATATAGTGCTGTG-3′ (SEQ ID NO: 5)) followed by FACS sorting to select GFP positive cells 48 hours post-transfection. Single clones were isolated and validated by immunoblotting for Rab27a, and two clones were pooled to establish the Rab27a-deficient cell line (9464D crRab27a).

### Immunoblotting

Cells were lysed in RIPA lysis buffer containing protease inhibitor cocktail (Sigma-Aldrich, P8340). Whole cell lysate and sEV protein concentrations were determined using a Pierce BCA Protein Assay Kit (VWR Scientific, PI23225) according to manufacturer's instructions. Membranes were blocked in Intercept Blocking Buffer (LI-COR Biosciences, 927-66003) followed by primary antibody incubation at 4° C. overnight. Membranes were washed, incubated with IR-conjugated secondary antibodies for one hour at room temperature (LI-COR Biosciences, 925-32213 and 925-68072) and imaged using the LI-COR Odyssey CLx Imager.

### Transmission Electron Microscopy (TEM)

Microscopy of sEVs was preformed according to a previously described method.²⁶ Briefly, 10 μL of the sEV sample was loaded onto a 400-mesh copper grid with carbon-coated formvar film and incubated for two minutes. The grid was briefly placed on 10 μL of 2% uranyl acetate for two minutes, followed by blotting to remove excess liquid. The grid was allowed to dry and imaged using a JEOL JEM1400 Transmission Electron Microscope (JEOL USA Inc.).

### Nanoparticle Tracking Analysis (NTA)

sEV samples were diluted to 1 mL (1:1000) with particle-free water. Each sample was loaded by syringe pump into the NanoSight NS300 (Malvern Instruments Ltd) set in scatter mode, and five 60-second videos were generated. The size distribution and concentration of particles were analyzed, and images were acquired using NanoSight software version 3.2 (Malvern Instruments Ltd.). To quantify in vitro sEV secretion from neuroblastoma cells upon tipifarnib treatment, cells were treated with DMSO or 0.1 M tipifarnib in sEV-depleted media for 48 hours. Cells were trypsinized using 0.05% Trypsin-EDTA (VWR Scientific, 45000-660) at the time of conditioned media collection and counted using a Cellometer Auto 2000 Cell Viability Counter (Nexcelom Bioscience). The corresponding sEV pellets were resuspended in 3 μL PBS per 10⁶ cells.

Flow Cytometric Analysis of sEVs

Flow cytometric analysis of sEVs was performed using a protocol adapted from Marimpietr et al. (45) Briefly, 5 μg of purified sEVs was added to 10 μL of 4 μm aldehyde/sulfate latex beads (Thermo Fisher, A37304) and incubated for at least 2 hours at room temperature. Twenty μL of 2% FBS in PBS was added to each sample and further incubated for 1 hour at room temperature. The sEV-coated beads were washed with PBS (1 mL) and pelleted at 3,000 RCF for 5 minutes at room temperature. The pellet containing sEV-coated beads was re-suspended in 200 μL of 100 mM glycine in PBS, incubated for 30 minutes at room temperature and pelleted by centrifugation at 3,000 RCF for 5 minutes. The sEV-coated beads were washed twice with 2% FBS in PBS and incubated with FITC-conjugated anti-ganglioside GD2 (BioLegend, clone 14G2a, 1:25 dilution) antibodies in 100 μL FACS buffer for 30 minutes on ice. sEV-coated beads were washed twice with FACS buffer and resuspended in 0.5 mL FACS buffer for flow cytometric analysis.

### RNA Sequencing and Data Analysis

Tumors were isolated from mice receiving 5 treatments of dinutuximab or dinutuximab plus sEVs (dosed twice per week as described in the Animal Experiments section). Tumor tissue was flash frozen, and RNA was extracted using RNeasy Mini Kit (Qiagen, 74004). Two RNA samples isolated from tumors in each treatment group were submitted to Penn State College of Medicine Genomic Core Facility for library preparation and sequencing. cDNA libraries were generated according to the Illumina Stranded mRNA Prep pipeline (www.illumina.com/products/by-type/sequencing-kits/library-prep-kits/stranded-mrna-prep.html). The cDNA library was checked for size distribution using BioAnalyzer (Agilent). Final libraries were pooled, diluted to 2 nM and subsequently sequenced using the Illumina HiSeq 2500 platform. Adaptors were trimmed from raw FASTQ files using the BBDuk tool. The trimmed FASTQ files were mapped against the mm10 mouse reference genome using STAR aligner (physiology.med.cornell.edu/faculty/skrabanek/lab/angsd/lecture_notes/STARmanual.pdf). The aligned reads were quantified with htseq-count (htseq.readthedocs.io/en/master/count.html). The differential expression of gene (DEG) analysis was performed using Deseq2 (bioconductor.org/packages/release/bioc/html/DESeq2.html) R package. The cutoff is log 2FC >0.5 with an adjusted p-value (FDR)<0.1. Heatmaps were generated using ComplexHeatmap R package to visualize differential expressed genes. Gene set enrichment analysis was performed using fgsea R package with the Ontology (Biological Process) MSigDB gene set (C5: ontology gene sets, BP: GO Biological Process Ontology).

### Kaplan-Meier Survival Curves

The GSE62564 RNA-Seq dataset containing the gene expression profiles of 498 patient primary neuroblastoma tumors was used for generating Kaplan-Meier survival curve.(46) The Kaplan-Meier event-free survival curve was plotted using survminer R package (rpkgs.datanovia.com/survminer/). The total of 498 patient samples were evenly divided into three groups according to the expression level for the desired gene. Red, blue and green lines represent high expression (top 33%), median expression (intermediate 33%) and low expression (bottom 33%) groups.

### Tumor and Spleen Dissociation

Murine tumors and spleens were harvested at the experimental endpoint and mechanically disassociated. Tumors were further digested with tumor digestion buffer (1 g/mL Collagenase Type I (Thermo Fisher, 17018029) and 2000 Units/ml DNase (Sigma-Aldrich, DN25-100MG) in HBSS buffer at room temperature for 1 hour. Dissociated tumor cells and spleen cells were passed through a 70 μm cell strainer (Falcon BD, 352350), pelleted, and washed once with PBS supplemented with 1% FBS. Red blood cells were lysed with RBC lysis buffer (0.0155 M NH₄Cl, 1.2 mM NaHCO₃, 0.01 mM Na₂EDTA, pH 7.4 in distilled water) for 3 minutes on ice and neutralized with PBS supplemented with 10% FBS. Cells were centrifuged and resuspended in FACS buffer to obtain single cell suspensions.

In Vivo Uptake of Neuroblastoma-Derived sEVs

In vivo uptake of neuroblastoma-derived sEVs was performed according to a previously described method.(19) Briefly, sEVs isolated from 9464D-GD2 cells were labeled with 1 mM Vybrant DiD Cell Labeling Solution (Thermo Fisher, V22887) at room temperature for 10 minutes, pelleted by centrifugation at 120,000 RCF for 2 hours at 4° C., and washed once in PBS. Vybrant DiD-labeled pellets were resuspended in PBS and injected (30 g in 100 μL PBS) via the tail vein into C57BL/6 mice (8- to 10-week-old). At 6, 12, and 24 hours after injection, mice were euthanized, whole organs isolated, and fluorescent IVIS imaging was performed. Radiant efficiency was calculated using ROI measurements of each organ.

9464D crRab27a Animal Experiment

C57BL/6 mice (8- to 10-week-old, both male and female) were subcutaneously injected with 1×10⁶ 9464D-GD2 cells or 4×10⁶ 9464D crRab27a cells in a 50:50 mixture of DMEM and Matrigel (Corning). After 18 Days, mice were euthanized, and tumors were harvested for analysis.

### Cell Viability and Proliferation Assays

9464D-GD2 cells were seeded in a white, opaque 96-well plate. The following day, cells were treated with increasing doses of tipifarnib, and cell viability was measured at 24, 48 and 72 hours post-treatment using the CellTiter-Glo® Luminescent Cell Viability Assay (Promega, G7573) according to manufacturer's instructions. Microplate luminescence was measured using a CLARIOstar (BMG Labtech) plate reader. All data were normalized to non-treated controls. 9464D cells or 9464D crRab27a cells were plated at a density of 5000 cells/well in a 96-well plate and monitored for confluence using the Incucyte S3 μLive Cell Imaging System (Sartorius). The percent cell confluence was quantified using Incucyte Basic Cell Analysis package (Sartorius).

sEV Isolation from Mouse Serum

For serum preparation, blood was collected using Microtainer Capillary Blood Collectors (Fisher) and centrifuged for 5 minutes at 500 RCF and 4° C. The supernatant was transferred to a 1.5 mL Eppendorf tube and centrifuged at 2,000 RCF for 15 minutes at 4° C. The supernatant (serum) was collected in a new 1.5 mL Eppendorf tube and stored at −80° C. for sEV isolation. For serum sEV isolation, serum samples were thawed on ice and 3-4 samples (equal volume) from each treatment group were pooled (500 μL total volume). The pooled serum sample was centrifuged at 20,000 RCF for 20 minutes at 4° C. to remove microvesicles. The supernatant was centrifuged at 120,000 RCF for 4 hours at 4° C. to pellet sEVs. The sEV-containing pellet was washed with PBS and centrifuged again at 120,000 RCF for 4 hours at 4° C. sEVs were resuspended in 250 μL PBS and stored at −20° C. prior to further analysis. Serum sEV protein concentration was determined using a Pierce BCA Protein Assay Kit (VWR Scientific PI23225) according to manufacturer's instructions. Serum sEV vesicle number was quantified by NTA.

### Cell Lines and Plasmids

The mouse neuroblastoma cell line 9464D was a gift from Dr. Paul Sondel (University of Wisconsin, Madison, WI). Human neuroblastoma BIR32 (CCL-127), HEK 293T/17 (CRL-11268) and NK92-EGFP-CD16 (PTA-8836) cell lines were purchased from ATCC. Neuroblastoma and HEK 293T/17 cells were cultured in Dulbecco's Modified Eagle's Medium (DMEM; Corning, 10-013-CV) supplemented with 10% heat-inactivated FBS (Sigma-Aldrich F2442) and 1% antibiotic-antimycotic (Corning, 30-004-CI). NK-92-EGFP-CD16 cells were cultured in RPMI 1640 (Corning, 10-040-CV) containing 10% horse serum (Equitech-Bio, SE30-0100), 10% heat-inactivated FBS, and 100 units/mL TL-2 (BioLegend, 589104-BL). Cells were incubated at 37° C. in a humidified chamber containing 5% CO₂. All cell lines in the lab are passaged for less than 6 months before use and periodically authenticated by Mycoplasma testing, morphologic inspection, and STR analysis.

The pCDH1-CMV-MCS-SV40-Hygro construct was previously described (18). SFG.GD3synthase (St8sial)-2A-GD2synthase (B4galnt1) was obtained from Martin Pule through Addgene (#75013). pCDH1-CMV-St8sia1-2A-B4galnt1-SV40-Hygro construct was generated by subcloning the PCR amplified (primer set comprising the primer: 5′-ATCCTCTAGACTGCCACCATGAG-3′ (SEQ ID NO: 1), and 5′-TAAATTCGAATCACTCGGCGGTCATGCACT-3′ (SEQ ID NO: 2) St8sial-2A-B4galnt1 cassette into the XbaI-BstBI site of pCDH1-CMV-MCS-SV40-Hygro. 9464D-GD2 cells were generated by transducing 9464D cells with lentiviral particles harboring pCDH1-CMV-St8sial-2A-B4galnt1-SV40-Hygro followed by selection with hygromycin (300 μg/mL).

### Drugs and Antibodies

Dinutuximab (Unituxin®) was a gift from Penn State Health Pharmacy (Hershey, PA, USA). Tipifarnib (AdooQ, MedChemExpress) was dissolved in DMSO to create a 10 mM stock solution for in vitro or suspended at 4 mg/mL in 20% w/v hydroxypropyl-p-cyclodextrin (Millipore Sigma, 332607-100G) in distilled water, pH 2.5 for in vivo studies. The following antibodies were used for immunoblotting: Alix (Cell Signaling Technology, 3A9, 1:1000), CD63 (Abcam, ab217345, 1:1000), Calnexin (Abcam, ab22595, 1:1000), Tsg101 (GeneTex, 70255, 1:500), Rab27a (Cell Signaling Technology, 69295S, 1:1000), j-Actin (Sigma, A5441, 1:10000) and Golgin97 (Thermo Fisher, A-21270, 1:1000). Antibodies used for flow cytometry are included in Table 1.

### Isolation of Small Extracellular Vesicles

sEVs were isolated from conditioned cell culture medium according to a previously described differential ultracentrifugation method (19). Briefly, FBS was depleted of sEVs by centrifuging heat-inactivated FBS twice at 120,000 RCF for 12 hours at 4° C. (Beckman, SW32Ti) followed by filtration of the supernatant through a 0.2-μm filter. Conditioned cell culture medium was collected from cells cultured for 24 hours in DMEM supplemented with 10% sEV-depleted FBS. The conditioned medium was centrifuged at 500 RCF for 10 minutes at 4° C. (Beckman, SX4750A) to remove cells and large cell debris. The supernatant filtered through a 0.2-μm syringe filter (VWR 28145-501) and concentrated using a 100K MWCO protein concentrator (Thermo Fisher, 88533). The concentrated supernatant was centrifuged at 10,000 RCF for 20 minutes at 4° C. (Eppendorf, FA-45-30-11) to remove larger microvesicles and apoptotic bodies followed by centrifugation at 120,000 RCF for 4 hours at 4° C. (Beckman, SW55Ti). The sEV-containing pellet was washed twice in ice-cold PBS and pelleted by centrifugation at 120,000 RCF at 4° C. for 4 hours and 12 hours, respectively (Beckman, SW55Ti). The sEV-containing pellet was resuspended in PBS and stored at −20° C.

### Animal Experiments

All animal studies were performed according to guidelines established by the Institutional Animal Care and Use Committee (IACUC) at the Penn State College of Medicine (Hershey, PA). An immunocompetent mouse model of neuroblastoma was generated by subcutaneously injecting 1×10⁶ 9464D-GD2 cells in a 50:50 mixture of DMEM and Matrigel (Corning, 354234) into C57BL/6J mice (8- to 10-week-old, JAX 000664) with male and female mice represented at an equal ratio. One week following tumor cell inoculation, mice were randomized into treatment groups. Where indicated, mice were treated twice-weekly by tail-vein injection with PBS (100 μL), dinutuximab (25 g in 100 μL PBS), purified sEVs from 9464D-GD2 cells (20 g in 100 μL PBS), or the combination of dinutuximab and sEVs. Where indicated, tipifarnib (25 mg/kg) or an equivalent volume of vehicle was administered twice daily by oral gavage. Primary tumor growth was monitored by measuring tumor volume using calipers (volume=π(length*width²)/6). At the experimental endpoint, mice were euthanized, and tumor, blood, spleen and bone marrow were harvested for ex vivo analysis. Endpoint tumor volume was calculated by measuring tumors using calipers (volume=π(length*width*height)/6).

### Flow Cytometry

For cell surface staining, approximately 1×10⁶ cells were stained with pre-mixed antibody cocktail panels (Table 1) in 100 μL FACS buffer (1% FBS, 0.2% NaN₃ in PBS) for 30 minutes on ice. Cells were washed twice with FACS buffer and fixed with 2% paraformaldehyde (Fisher Scientific, 50-980-487) for 15 minutes. Fixed cells were washed once with PBS and resuspended in 0.5 mL FACS buffer for flow cytometric analysis using a LSR II or Symphony flow cytometer (BD Biosciences). Compensation was performed using UltraComp eBeads™ compensation beads (Invitrogen). FlowJo™ v10 (FlowJo, LLC) software was used for data analysis.

### In Vitro NK Cell-Mediated ADCC Assay

The co-culture assay was adapted and modified from Barry, et al (20). Neuroblastoma cells were seeded at 2×10⁴ cells per well in a 96-well plate. The next day, the medium was removed, cells were washed in PBS and 1×10⁴ NK92-CD16-EGFP cells (effector:target ratio of 1:2) were added in RPMI 1640 containing 10% FBS, 100 units/mL IL2, and 0.5 μM YOYO-3 iodide (ThermoFisher, Y3606). Where indicated, NK92-CD16-EGFP cells were pre-incubated with neuroblastoma-derived sEVs (25 μg/mL) for two hours prior to addition, and dinutuximab was added to the co-culture at a concentration of 100 ng/mL. Images were taken at one-hour intervals using the Incucyte S3 μLive Cell Imaging System (Sartorius) and quantified using the Incucyte Cell-by-Cell Analysis Software Module (Sartorius).

### Statistical Analysis

GraphPad Prism (GraphPad Software, Inc.) was used for statistical analysis. Two-tailed unpaired student t-tests were used for single comparisons. One-way ANOVA with Tukey's/Sidak's post hoc tests were used for multiple comparisons. Statistical significance was set top <0.05.

Unless defined otherwise, all technical and scientific terms used herein have the same meanings as commonly understood by one of skill in the art to which the disclosed invention belongs. Publications cited herein and the materials for which they are cited are specifically

### INCORPORATED BY REFERENCE

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

TABLE 1
Flow cytometry antibody panels.

| Panel | Antibody | Clone | Source | Dilution |
|---|---|---|---|---|
| GD2 expression | FITC anti-human Ganglioside | 14G2a | BioLegend | 1:25 |
| | GD2 Antibody #357314 | | | |
| Tumor | Alexa Fluor ® 700 anti-mouse | 30-F11 | BioLegend | 1:250 |
| infiltrating | CD45 Antibody #103128 | | | |
| immune cells | PE anti-mouse CD3 Antibody #100206 | 17A2 | BioLegend | 1:100 |
| | Brilliant Violet 421 ™ anti-mouse | PK136 | BioLegend | 1:100 |
| | NK-1.1 Antibody #108741 | | | |
| | APC anti-mouse/human CD11b | M1/70 | BioLegend | 1:100 |
| | Antibody #101212 | | | |
| | FITC anti-mouse F4/80 Antibody #123108 | BM8 | BioLegend | 1:250 |
| Spleen NK cell | PE anti-mouse CD45 Antibody #103106 | 30-F11 | BioLegend | 1:100 |
| subpopulations | FITC anti-mouse CD3 Antibody #100204 | 17A2 | BioLegend | 1:125 |
| | FITC anti-mouse CD19 Antibody #115506 | 6D5 | BioLegend | 1:200 |
| | Brilliant Violet 421 ™ anti-mouse NK-1.1 | PK136 | BioLegend | 1:100 |
| | Antibody #108741 | | | |
| | APC anti-mouse/human CD11b Antibody | M1/70 | BioLegend | 1:100 |
| | #101212 | | | |
| | PE/Cyanine7 anti-mouse/rat/human CD27 | LG.3A10 | BioLegend | 1:100 |
| | Antibody #124216 | | | |
| BM myeloid | PE anti-mouse CD45 Antibody #103106 | 30-F11 | BioLegend | 1:100 |
| cell | FITC anti-mouse F4/80 Antibody #123108 | BM8 | BioLegend | 1:250 |
| subpopulations | APC anti-mouse/human CD11b Antibody | M1/70 | BioLegend | 1:100 |
| | #101212 | | | |
| | Brilliant Violet 421 ™ anti-mouse Ly-6C | HK1.4 | BioLegend | 1:25 |
| | Antibody #128032 | | | |
| | Alexa Fluor ® 700 anti-mouse Ly-6G | 1A8 | BioLegend | 1:200 |
| | Antibody #127622 | | | |
| Blood NK cells | PE anti-mouse CD45 Antibody #103106 | 30-F11 | BioLegend | 1:100 |
| | FITC anti-mouse CD3 Antibody #100204 | 17A2 | BioLegend | 1:125 |
| | FITC anti-mouse CD19 Antibody #115506 | 6D5 | BioLegend | 1:200 |
| | Brilliant Violet 421 ™ anti-mouse NK-1.1 | PK136 | BioLegend | 1:100 |
| | Antibody #108741 | | | |

TABLE 2
Sequences

| SEQ ID NO: | Description | Sequence |
|---|---|---|
| SEQ ID NO: 1 | Primer for St8sial- | 5′- ATCCTCTAGACTGCCACCATGAG-3′ |
| | 2A-B4galnt1 | |
| | cassette | |
| SEQ ID NO: 2 | Primer for St8sial- | 5′- TAAATTCGAATCACTCGGCGGTCATGCACT-3′ |
| | 2A-B4galnt1 | |
| | cassette | |
| SEQ ID NO: 3 | Primer for murine | 5′-TGGTTAAGCTACGAAACCTA-3′ |
| | Rab27a | |
| SEQ ID NO: 4 | Primer for murine | 5′-AGTGTACTGGTAGAGTACAC-3′ |
| | Rab27a | |
| SEQ ID NO: 5 | Primer for murine | 5′-AACCCAGATATAGTGCTGTG-3′ |
| | Rab27a | |

### REFERENCES

1. Irwin M S, Park J R. Neuroblastoma: paradigm for precision medicine. Pediatr Clin North Am 2015; 62:225-56
2. Matthay K K, Maris J M, Schleiermacher G, Nakagawara A, Mackall C L, Diller L, et al. Neuroblastoma. Nat Rev Dis Primers 2016; 2:16078
3. Wienke J, Dierselhuis M P, Tytgat G A M, Kunkele A, Nierkens S, Molenaar J J. The immune landscape of neuroblastoma: Challenges and opportunities for novel therapeutic strategies in pediatric oncology. Eur J Cancer 2021; 144:123-50
4. Casey D L, Cheung N V. Immunotherapy of Pediatric Solid Tumors: Treatments at a Crossroads, with an Emphasis on Antibodies. Cancer Immunol Res 2020; 8:161-6
5. Cheung N K, Saarinen U M, Neely J E, Landmeier B, Donovan D, Coccia P F. Monoclonal antibodies to a glycolipid antigen on human neuroblastoma cells. Cancer Res 1985; 45:2642-9
6. Nazha B, Inal C, Owonikoko T K. Disialoganglioside G D2 Expression in Solid Tumors and Role as a Target for Cancer Therapy. Front Oncol 2020; 10:1000
7. Sait S, Modak S. Anti-GD2 immunotherapy for neuroblastoma. Expert Rev Anticancer Ther 2017; 17:889-904
8. Keyel M E, Reynolds C P. Spotlight on dinutuximab in the treatment of high-risk neuroblastoma: development and place in therapy. Biologics 2019; 13:1-12
9. Ahmed M, Cheung N K. Engineering anti-GD2 monoclonal antibodies for cancer immunotherapy. FEBS Lett 2014; 588:288-97
10. Olejarz W, Dominiak A, Zolnierzak A, Kubiak-Tomaszewska G, Lorenc T. Tumor-Derived Exosomes in Immunosuppression and Immunotherapy. J Immunol Res 2020; 2020:6272498
11. Gao Y, Qin Y, Wan C, Sun Y, Meng J, Huang J, et al. Small Extracellular Vesicles: A Novel Avenue for Cancer Management. Front Oncol 2021; 11:638357
12. Chen G, Huang A C, Zhang W, Zhang G, Wu M, Xu W, et al. Exosomal PD-L1 contributes to immunosuppression and is associated with anti-PD-1 response. Nature 2018; 560:382-6
13. Poggio M, Hu T, Pai C C, Chu B, Belair C D, Chang A, et al. Suppression of Exosomal PD-L1 Induces Systemic Anti-tumor Immunity and Memory. Cell 2019; 177:414-27.e13
14. Capuano C, Pighi C, Battella S, De Federicis D, Galandrini R, Palmieri G. Harnessing CD16-Mediated NK Cell Functions to Enhance Therapeutic Efficacy of Tumor-Targeting mAbs. Cancers (Basel) 2021; 13
15. Battke C, Ruiss R, Welsch U, Wimberger P, Lang S, Jochum S, et al. Tumour exosomes inhibit binding of tumour-reactive antibodies to tumour cells and reduce ADCC. Cancer Immunol Immunother 2011; 60:639-48
16. Berchem G, Noman M Z, Bosseler M, Paggetti J, Baconnais S, Le Cam E, et al. Hypoxic tumor-derived microvesicles negatively regulate NK cell function by a mechanism involving TGF-0 and miR23a transfer. Oncoimmunology 2016; 5:e1062968
17. Zhao J, Schlößer HA, Wang Z, Qin J, Li J, Popp F, et al. Tumor-Derived Extracellular Vesicles Inhibit Natural Killer Cell Function in Pancreatic Cancer. Cancers (Basel) 2019; 11
18. Takahashi Y, Liang X, Hattori T, Tang Z, He H, Chen H, et al. VPS37A directs ESCRT recruitment for phagophore closure. J Cell Biol 2019; 218:3336-54
19. Wills C A, Liu X, Chen L, Zhao Y, Dower C M, Sundstrom J, et al. Chemotherapy-Induced Upregulation of Small Extracellular Vesicle-Associated PTX3 Accelerates Breast Cancer Metastasis. Cancer Res 2021; 81:452-63
20. Barry W E, Jackson J R, Asuelime G E, Wu H W, Sun J, Wan Z, et al. Activated Natural Killer Cells in Combination with Anti-GD2 Antibody Dinutuximab Improve Survival of Mice after Surgical Resection of Primary Neuroblastoma. Clin Cancer Res 2019; 25:325-33
21. Kroesen M, Brok I C, Reijnen D, van Hout-Kuijer M A, Zeelenberg I S, Den Brok M H, et al. Intra-adrenal murine TH-MYCN neuroblastoma tumors grow more aggressive and exhibit a distinct tumor microenvironment relative to their subcutaneous equivalents. Cancer Immunol Immunother 2015; 64:563-72
22. Kroesen M, Nierkens S, Ansems M, Wassink M, Orentas R J, Boon L, et al. A transplantable TH-MYCN transgenic tumor model in C57Bl/6 mice for preclinical immunological studies in neuroblastoma. Int J Cancer 2014; 134:1335-45
23. Voeller J, Erbe A K, Slowinski J, Rasmussen K, Carlson P M, Hoefges A, et al. Combined innate and adaptive immunotherapy overcomes resistance of immunologically cold syngeneic murine neuroblastoma to checkpoint inhibition. J Immunother Cancer 2019; 7:344
24. Théry C, Witwer K W, Aikawa E, Alcaraz M J, Anderson J D, Andriantsitohaina R, et al. Minimal information for studies of extracellular vesicles 2018 (MISEV2018): a position statement of the International Society for Extracellular Vesicles and update of the MISEV2014 guidelines. J Extracell Vesicles 2018; 7:1535750
25. Flores-Toro J A, Luo D, Gopinath A, Sarkisian M R, Campbell J J, Charo I F, et al. CCR2 inhibition reduces tumor myeloid cells and unmasks a checkpoint inhibitor effect to slow progression of resistant murine gliomas. Proc Natl Acad Sci USA 2020; 117:1129-38
26. Heger L, Balk S, Luhr J J, Heidkamp G F, Lehmann C H K, Hatscher L, et al. CLEC10A Is a Specific Marker for Human CD1c. Front Immunol 2018; 9:744
27. Thebault P, Lhermite N, Tilly G, Le Texier L, Quillard T, Heslan M, et al. The C-type lectin-like receptor CLEC-1, expressed by myeloid cells and endothelial cells, is up-regulated by immunoregulatory mediators and moderates T cell activation. J Immunol 2009; 183:3099-108
28. Lewis S M, Williams A, Eisenbarth S C. Structure and function of the immune system in the spleen. Sci Immunol 2019; 4
29. Chiossone L, Chaix J, Fuseri N, Roth C, Vivier E, Walzer T. Maturation of mouse NK cells is a 4-stage developmental program. Blood 2009; 113:5488-96
30. Riggan L, Shah S, O'Sullivan T E. Arrested development: suppression of NK cell function in the tumor microenvironment. Clin Transl Immunology 2021; 10:e1238
31. Peinado H, Aleckovid M, Lavotshkin S, Matei I, Costa-Silva B, Moreno-Bueno G, et al. Melanoma exosomes educate bone marrow progenitor cells toward a pro-metastatic phenotype through MET. Nat Med 2012; 18:883-91
32. Datta A, Kim H, McGee L, Johnson A E, Talwar S, Marugan J, et al. High-throughput screening identified selective inhibitors of exosome biogenesis and secretion: A drug repurposing strategy for advanced cancer. Sci Rep 2018; 8:8161
33. Shand F H, Ueha S, Otsuji M, Koid S S, Shichino S, Tsukui T, et al. Tracking of intertissue migration reveals the origins of tumor-infiltrating monocytes. Proc Natl Acad Sci USA 2014; 111:7771-6
34. Movahedi K, Laoui D, Gysemans C, Baeten M, Stangé G, Van den Bossche J, et al. Different tumor microenvironments contain functionally distinct subsets of macrophages derived from Ly6C(high) monocytes. Cancer Res 2010; 70:5728-39

35. Bronte V, Brandau S, Chen S H, Colombo M P, Frey A B, Greten T F, et al. Recommendations for myeloid-derived suppressor cell nomenclature and characterization standards. Nat Commun 2016; 7:12150
36. Melaiu O, Chierici M, Lucarini V, Jurman G, Conti L A, De Vito R, et al. Cellular and gene signatures of tumor-infiltrating dendritic cells and natural-killer cells predict prognosis of neuroblastoma. Nat Commun 2020; 11:5992
37. Asgharzadeh S, Salo J A, Ji L, Oberthuer A, Fischer M, Berthold F, et al. Clinical significance of tumor-associated inflammatory cells in metastatic neuroblastoma. J Clin Oncol 2012; 30:3525-32
38. Webb E R, Lanati S, Wareham C, Easton A, Dunn S N, Inzhelevskaya T, et al. Immune characterization of pre-clinical murine models of neuroblastoma. Sci Rep 2020; 10:16695
39. Li I, Nabet B Y. Exosomes in the tumor microenvironment as mediators of cancer therapy resistance. Mol Cancer 2019; 18:32
40. Hong C S, Sharma P, Yemeni S S, Simms P, Jackson E K, Whiteside T L, et al. Circulating exosomes carrying an immunosuppressive cargo interfere with cellular immunotherapy in acute myeloid leukemia. Sci Rep 2017; 7:14684
41. Zhang H, Lu J, Liu J, Zhang G, Lu A. Advances in the discovery of exosome inhibitors in cancer. J Enzyme Inhib Med Chem 2020; 35:1322-30
42. Gilardi M, Wang Z, Proietto M, Chilli A, Calleja-Valera J L, Goto Y, et al. Tipifarnib as a Precision Therapy for. Mol Cancer Ther 2020; 19:1784-96
43. Ackermann S, Cartolano M, Hero B, Welte A, Kahlert Y, Roderwieser A, et al. A mechanistic classification of clinical phenotypes in neuroblastoma. Science 2018; 362:1165-70
44. Eleveld T F, Oldridge D A, Bernard V, Koster J, Colmet Daage L, Diskin S J, et al. Relapsed neuroblastomas show frequent RAS-MAPK pathway mutations. Nat Genet 2015; 47:864-71
45. Marimpietri D, Petretto A, Raffaghello L, Pezzolo A, Gagliani C, Tacchetti C, et al. Proteome profiling of neuroblastoma-derived exosomes reveal the expression of proteins potentially involved in tumor progression. PLoS One 2013; 8:e75054
46. Su Z, Fang H, Hong H, Shi L, Zhang W, Zhang Y, et al. An investigation of biomarkers derived from legacy microarray data for their utility in the RNA-seq era. Genome Biol 2014; 15:523

1. A composition comprising an anti-GD2 immunotherapy and a farnesyltransferase inhibitor.

2. The composition of claim 1, wherein the farnesyltransferase inhibitor comprises tipifarnib.

3. The composition of claim 1, wherein the anti-GD2 immunotherapy comprises dinutuximab.

4. A method of treating a cancer comprising administering to a subject in need thereof an anti-GD2 immunotherapy and a farnesyltransferase inhibitor.

5. The method of claim 4, wherein the cancer is neuroblastoma.

6. The method of claim 4, wherein the cancer is high-risk neuroblastoma.

7. The method of claim 4, wherein the anti-GD2 immunotherapy is dinutuximab.

8. The method of claim 4, wherein the farnesyltransferase inhibitor is tipifarnib.

9. The method of claim 4, further comprising administering a therapeutically effective amount of at least one or more active agents or a support care therapy selected from the group comprising: a DNA-hypomethylating agent, therapeutic antibody that specifically binds to a cancer antigen, a hematopoietic growth factor, a cytokine, an antibiotic, a cox-2 inhibitor, an immunomodulatory agent, an anti-thymocyte globulin, an immunosuppressive agent, and a corticosteroid.

10. The method of claim 4, wherein the administration of the therapeutically effective amount of the anti-GD2 immunotherapy and the farnesyltransferase inhibitor provides an increase in immune cell tumor infiltration, a decrease in tumor-associate macrophages, an increase in tumor-infiltrating natural killer (NK) cells, an increase in splenic NK cell maturation, an increase in NK cell-mediated antibody-dependent cellular cytotoxicity, a decrease of immunosuppressive effects of neuroblastoma-derived sEVs, or a decrease of formation of CD11b+Ly6C(high)Ly6G(low) cells.

11. The method of claim 4, wherein the subject comprises one or more cell markers corresponding to low expression of NKG2D and/or RAS/MAPK signal pathway deficiencies.

12. The method of claim 5, wherein the neuroblastoma is a GD2 inhibitor-refractory neuroblastoma (GIRN).

13. A method of preventing neuroblastoma metastasis, comprising administering to a subject a therapeutically effective amount of an anti-GD2 immunotherapy and a farnesyltransferase inhibitor.

14. The method of claim 13, wherein the anti-GD2 immunotherapy is dinutuximab.

15. The method of claim 13, wherein the farnesyltransferase inhibitor is tipifarnib.

16. The method of claim 13, further comprising administering a therapeutically effective amount of at least one or more active agents or a support care therapy selected from the group consisting of a DNA-hypomethylating agent, therapeutic antibody that specifically binds to a cancer antigen, a hematopoietic growth factor, a cytokine, an antibiotic, a cox-2 inhibitor, an immunomodulatory agent, an anti-thymocyte globulin, an immunosuppressive agent, and a corticosteroid.

17. The method of claim 13, wherein the administration of the therapeutically effective amount of the anti-GD2 immunotherapy and the farnesyltransferase inhibitor provides an increase in immune cell tumor infiltration, a decrease in tumor-associate macrophages, an increase in tumor-infiltrating natural killer (NK) cells, an increase in splenic NK cell maturation, an increase in NK cell-mediated antibody-dependent cellular cytotoxicity, a decrease of immunosuppressive effects of neuroblastoma-derived sEVs, or a decrease of formation of CD11b+Ly6C(high)Ly6G(low) cells.


SEQUENCE LISTING (5 sequences)

SEQ ID NO: 1 - DNA, 23 bp, synthetic construct
  source 1..23: mol_type = other DNA
atcctctagactgccaccatgag

SEQ ID NO: 2 - DNA, 30 bp, synthetic construct
  source 1..30: mol_type = other DNA
taaattcgaatcactcggcggtcatgcact

SEQ ID NO: 3 - DNA, 20 bp, synthetic construct
  source 1..20: mol_type = other DNA
tggttaagctacgaaaccta

SEQ ID NO: 4 - DNA, 20 bp, synthetic construct
  source 1..20: mol_type = other DNA
agtgtactggtagagtacac

SEQ ID NO: 5 - DNA, 20 bp, synthetic construct
  source 1..20: mol_type = other DNA
aacccagatatagtgctgtg